SEQUENCE LISTING (17 sequences)

SEQ ID NO: 1 - DNA, 3258 bp, Artificial Sequence
  IBDV strain R903/78 segment A
ggatacgatcggtctgaccccgggggagtcacccggggacaggccgtcaaggctttgttccaggatggaactcctccttctacaacgctatcattgatggtcagtagagatcagacaaacgatcgcagcgatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagttacaactactgcaggctagtgagtcggagtctcacagtgaggtcaagcacacttcctggtggcgtttatgcactaaacggcaccataaacgccgtgaccttccaaggaagcctgagtgaactgacagatgttagctacaatgggttgatgtctgcaacagccaacatcaacgacaaaattgggaacgtcctagtaggggaaggggtcaccgtcctcagcttacccacatcatatgatcttgggtatgtgaggcttggtgaccccattcccgcaatagggcttgacccaaaaatggtagccacatgtgacagcagtgacaggcccagagtctacaccataactgcagccgatgattaccaattctcatcacagtaccaaccaggtggggtaacaatcacactgttctcagccaacattgatgccatcacaagcctcagcgttgggggagagctcgtgtttcaaacaagcgtccacggcattgtactgggcgccaccatctacctcataggctttgatgggacagcggtaatcaccagggctgtggccgcaaacaatgggctgacgaccggcaccgacaaccttatgccattcaatcttgtgattccaacaaacgagataacccagccaatcacatccatcaaactggagatagtgacctccaaaagtggtggtcaggcaggggatcagatgtcatggtcggcaagagggagcctagcagtgacgatccatggtggcaactatccaggggccctccgtcccgtcacgctagtggcctacgaaagagtggcaacaggatccgtcgttacggtcgctggggtgagcaacttcgagctgatcccaaatcctgaactagcaaagaacctggttacagaatacggccgatttgacccaggagccatgaactacacaaaattgatactgagtgagagggaccgtcttggcatcaagaccgtctggccaacaagggagtacactgactttcgtgaatacttcatggaggtggccgacctcaactctcccctgaagattgcaggagccttcggcttcaaagacataatccgggccataaggaggatagctgtgccggtggtctccacattgttcccacctgccgctcccctagcccatgcaattggggaaggtgtagactacctgctgggcgatgaggcacaggctgcttcaggaactgctcgagccgcgtcaggaaaagcaagagctgcctcaggccgcataaggcagctgactctcgccgccgacaaggggtacgaggtagtcgcgaatctattccaggtgccccagaatcccgtagtcgacgggattcttgcttcacctggggtactccgcggtgcacacaacctcgactgcgtgttaagagagggtgccacgctattccctgtggttattacgacagtggaagacgccatgacacccaaagcattgaacagcaaaatgtttgctgtcattgaaggcgtgcgagaagacctccaacctccatctcaaagaggatccttcatacgaactctctctggacacagagtctatggatatgctccagatggggtacttccactggagactgggagagactacaccgttgtcccaatagatgatgtctgggacgacagcattatgctgtccaaagatcccatacctcctattgtgggaaacagtggaaatctagccatagcttacatggatgtgtttcgacccaaagtcccaatccatgtggctatgacgggagccctcaatgcttgtggcgagattgagaaagtaagctttagaagcaccaagctcgccactgcacaccgacttggccttaagttggctggtcccggagcattcgatgtaaacaccgggcccaactgggcaacgttcatcaaacgtttccctcacaatccacgcgactgggacaggctcccctacctcaacctaccataccttccacccaatgcaggacgccagtaccaccttgccatggctgcatcagagttcaaagagacccccgaactcgagagtgccgtcagagcaatggaagcagcagccaacgtggacccactattccaatctgcactcagtgtgttcatgtggctggaagagaatgggattgtgactgacatggccaacttcgcactcagcgacccgaacgcccatcggatgcgaaattttcttgcaaacgcaccacaagcaggcagcaagtcgcaaagggccaagtacgggacagcaggctacggagtggaggctcggggccccacaccagaggaagcacagagggaaaaagacacacggatctcaaagaagatggagaccatgggcatctactttgcaacaccagaatgggtagcactcaatgggcaccgagggccaagccccggccagctaaagtactggcagaacacacgagaaataccggacccaaacgaggactatctagactacgtgcatgcagagaagagccggttggcatcagaagaacaaatcctaagggcagctacgtcgatctacggggctccaggacaggcagagccaccccaagctttcatagacgaagttgccaaagtctatgaaatcaaccatggacgtggcccaaaccaagaacagatgaaagatctgctcttgactgcgatggagatgaagcatcgcaatcccaggcgggctctaccaaagcccaagccaaaacccaatgctccaacacagagaccccctggtcggctgggccgctggatcaggaccgtctctgatgaggaccttgagtgaggctcctgggagtctcccgacaccacccgcgcaggtgtggacaccaattcggccttacaacccaaattggatccgttcgcgggtcccct

SEQ ID NO: 2 - DNA, 2827 bp, Artificial Sequence
  IBDV strain R903/78 segment B
ggatacgatgggtttgaccctcagggagtcacgaattaacatggctactaggggcgatacccgccgctggctgccacgttagtggctcctcttcttgatgattctgccaccatgagtgacattttcaacagtccacaggcgcgaagcacgatctcagcagcgttcggcataaagcctactgctggacaagacgtggaagaactcttgatccctaaagtttgggtgccacctgaggatccgcttgccagccctagtcgactggcaaagttcctcagagagaacggctacaaagttttgcagccacggtctctgcccgagaatgaggagtatgagaccgaccaaatactcccagacttagcatggatgcgacagatagaaggggctgttttaaaacccactctatctctccctattggagatcaggagtacttcccaaagtactacccaacacatcgccctagcaaggagaagcccaatgcgtacccgccagacatcgcactactcaagcagatgatttacctgtttctccaggttccagaggccaacgagggcctaaaggatgaagtaaccctcttgacccaaaacataagggacaaggcctatggaagtgggacctacatgggacaagcaactcgacttgtggccatgaaggaggtcgccactggaagaaacccaaacaaggatcctctaaagcttgggtacacttttgagagcatcgcgcagctacttgacatcacactaccggtaggcccacccggtgaggatgacaagccctgggtgccactcacaagagtgccgtcacggatgttggtgctgacgggagacgtagatggcgactttgaggttgaagattaccttcccaaaatcaacctcaagtcatcaagtggactaccatatgtaggtcgcaccaaaggagagacaattggcgagatgatagctatctcaaaccagtttctcagagagctatcaacactgttgaagcaaggtgcagggacaaaggggtcaaacaagaagaagctactcagcatgttaagtgactattggtacttatcatgcgggcttttgtttccaaaggctgaaaggtacgacaaaagcacatggctcaccaagacccggaacatatggtcagctccatccccaacacacctcatgatctctatgatcacctggcccgtgatgtccaacagcccaaataacgtgttgaacattgaagggtgtccatcactctacaaattcaacccgttcagaggagggttgaacaggatcgtcgagtggatattggccccggaagaacccaaggctcttgtatatgcggacaacatatacattgtccactcaaacacgtggtactcaattgacctagagaagggtgaggcaaactgcactcgccaacacatgcaagccgcaatgtactacatactcaccagagggtggtcagacaacggcgacccaatgttcaatcaaacatgggccacctttgccatgaacattgcccctgctctagtggtggactcatcgtgcctgataatgaacctgcaaattaagacctatggtcaaggcagcgggaatgcagccacgttcatcaacaaccacctcttgagcacgctagtgcttgaccagtggaacctgatgagacagcccagaccagacagcgaggagttcaaatcaattgaggacaagctaggtatcaactttaagattgagaggtccattgatgatatcaggggcaagctgagacagcttgtcctccttgcacaaccagggtacctgagtgggggggttgaaccagaacaatccagcccaactgttgagcttgacctactagggtggtcagctacatacagcaaagatctcgggatctatgtgccggtgcttgacaaggaacgcctattttgttctgctgcgtatcccaagggagtagagaacaagagtctcaaatccaaagtcgggatcgagcaggcatacaaggtagtcaggtatgaggcgttgaggttggtaggtggttggaactacccactcctgaacaaagcctgcaagaataacgcaggcgccgctcggcggcatctggaggccaaggggttcccactcgacgagttcctagccgagtggtctgagctgtcagagttcggtgaggccttcgaaggcttcaatatcaagctgaccgtaacatctgagagcctagccgaactgaacaagccagtaccccccaagcccccaaatgtcaacagaccagtcaacactgggggactcaaggcagtcagcaacgccctcaagaccggtcggtacaggaacgaagccggactgagtggtctcgtccttctagccacagcaagaagccgtctgcaagatgcagttaaggccaaggcagaagccgagaaactccacaagtccaagccagacgaccccgatgcagactggttcgaaagatcagaaactctgtcagaccttctggagaaagccgacatcgccagcaaggtcgcccactcagcactcgtggaaacaagcgacgctcttgaagcagttcagtcgacttccgtgtacacccccaagtacccagaagtcaagaacccacagaccgcctccaaccccgttgttgggctccacctgcccgccaagagagccaccggtgtccaggccgctcttctcggagcaggaacgagcagaccaatggggatggaggccccaacacggtccaagaacgccgtgaaaatggccaaacggcggcaacgccaaaaggagagccgctaacagccatgatgggaaccactcaagaagaggacactaatcccagaccccgtatccccggccttcgcctgcgggggccccc

SEQ ID NO: 3 - DNA, 20 bp, Artificial Sequence
  IBDV_f primer
tcactacacactgcagagca

SEQ ID NO: 4 - DNA, 20 bp, Artificial Sequence
  IBDV_r primer
gagactccgactcactagcc

SEQ ID NO: 5 - DNA, 20 bp, Artificial Sequence
  IBDV_p
tgcccagaacctaccggcca

SEQ ID NO: 6 - DNA, 20 bp, Artificial Sequence
  IFN-beta_f primer
gacgccgcattgaccatcta

SEQ ID NO: 7 - DNA, 22 bp, Artificial Sequence
  IFN-beta_r primer
ccttaggatttccactctgact

SEQ ID NO: 8 - DNA, 21 bp, Artificial Sequence
  IFN-lambda_f primer
tgagctggccctgacgctgaa

SEQ ID NO: 9 - DNA, 19 bp, Artificial Sequence
  IFN-lambda_r primer
aggcggaggttgaaggtga

SEQ ID NO: 10 - DNA, 28 bp, Artificial Sequence
  IFN-gamma_f primer
gaaaagctgactaattattcggtaactg

SEQ ID NO: 11 - DNA, 22 bp, Artificial Sequence
  IFN-gamma_r primer
gttcagccatcacttggatgag

SEQ ID NO: 12 - DNA, 21 bp, Artificial Sequence
  ISG56_f primer
cctggagtactatgagcgggc

SEQ ID NO: 13 - DNA, 21 bp, Artificial Sequence
  IFG56_r primer
tgggtgcctaaggaccttgtc

SEQ ID NO: 14 - DNA, 20 bp, Artificial Sequence
  MxA_f primer
aggtcagttaccaggactac

SEQ ID NO: 15 - DNA, 20 bp, Artificial Sequence
  MxA_r primer
atggcattctgggctttatt

SEQ ID NO: 16 - DNA, 23 bp, Artificial Sequence
  GAPDH_f primer
ggtatcgtggaaggactcatgac

SEQ ID NO: 17 - DNA, 23 bp, Artificial Sequence
  GAPDH_r primer
atgccagtgagcttcccgttcag

(19) United States
(12) Patent Application Publication
(10) Pub. No.: US 2024/0299476 A1
(43) Pub. Date: Sep. 12, 2024

(54) USE OF A BIRNAVIRUS ALONE OR IN COMBINATION THERAPY FOR THE TREATMENT OF CANCER

(71) Applicant: Probiogen AG, Berlin (DE)

(72) Inventors: Tibor Bakacs, Budapest (HU); Volker Sandig, Berlin (DE); Shimon Slavin, Tel Aviv (IL); Sven Krugener, Berlin (DE); Wolfgang W. Renz, Rheinfelden (DE); Imre Kovesdi, Rockville, MD (US); Alexander Karlas, Berlin (DE); Deborah Horn, Berlin (DE)

(21) Appl. No.: 18/258,376

(22) PCT Filed: Dec. 15, 2021
(86) PCT No.: PCT/EP2021/085935

§ 371 Date: Jun. 20, 2023

(30) Foreign Application Priority Data
Dec. 21, 2020 (EP) 20216253.3

(51) Int. Cl. A61K 35/768; A61K 45/06; A61P 35/00; C12N 7/00

(52) U.S. Cl. CPC A61K 35/768; A61K 45/06; A61P 35/00; C12N 7/00; C12N 2720/10032

(57) ABSTRACT

The present invention relates to a birnavirus for use in the treatment or prevention of cancer. Further, the present invention relates to a combination comprising at least one birnavirus and at least one further active agent for use in the treatment or prevention of cancer. Furthermore, the present invention relates to a pharmaceutical composition comprising the birnavirus or the combination for use in the treatment or prevention of cancer.

The present invention relates to a birnavirus for use in the treatment or prevention of cancer. Further, the present invention relates to a combination comprising at least one birnavirus and at least one further active agent for use in the treatment or prevention of cancer. Furthermore, the present invention relates to a pharmaceutical composition comprising the birnavirus or the combination for use in the treatment or prevention of cancer.

### BACKGROUND OF THE INVENTION

Cancer is a generic term for a large group of diseases. One defining feature of cancer is the rapid creation of abnormal cells that grow beyond their usual boundaries. Prostate, colorectal, lung, liver and stomach cancer are the most common types of cancer in men, while breast, lung, colorectal, and cervical cancer are the most common among women. Cancer is the second leading cause of death: Not less than 1 in 6 deaths is due to cancer worldwide. Because cancer is a disease of aging, the number of cancer deaths worldwide is predicted to increase due to the increase proportion of elderly people especially in low- and middle-income countries where already today about 70% of the deaths from cancer are recorded. Furthermore, cancer is the disease which costs most. The economic impact of cancer is significant and will increase during the next two decades. Even if great progress has been made in the past decades in the prevention, detection, diagnosis, and treatment of cancer, some forms of cancer such as liver, lung, brain and pancreatic cannot be treated effectively.

A promising approach for fighting cancer cells resistant to conventional anti-cancer modalities, including cancer stem cells that are a priori resistant to chemotherapy and radiation therapy, is based on therapy concepts using cancer immunotherapy. Cancer immunotherapy approaches are based on down-regulation of negative regulators of the immune system (e.g. regulatory T cells; checkpoint inhibitors; myeloid-derived suppressor cells, etc.) and artificial stimulation of effector cells (e.g. T cells and natural killer (NK) cells) of the immune system, ideally also resulting in activation of antigen presenting cells of the immune system that lead to development of anti-cancer immunity (e.g. dendritic cells and macrophage s) which improves the immune system's natural ability to fight against cancer. While active cancer immunotherapy directs the immune system to attack cancer cells by targeting tumor markers, passive cancer immunotherapies enhances existing antitumor responses e.g. via stimulation with cytokines, lymphocytes, or monoclonal antibodies. Currently, T cells of the chimeric antigen receptor (CAR) and checkpoint inhibitors are promising approaches in cancer immunotherapy. For example, the combinatorial use of immunomodulatory medicinal products, using PD-1 and CTLA-4 blockade approaches on lymphocytes or on the cancer cells like Ipilimumab, Nivolumab, Pembrolizumab and Atezolizumab, Avelumab, Durvalumab, Tremlimumab, respectively, have been intensively investigated in more than 250 clinical studies.

Another promising approach for fighting cancer is based on therapy concepts using oncolytic viruses. An oncolytic virus is a virus that mainly kills cancer cells through infection. The oncolytic virus multiplies in infected cancer cells and releases new infectious virus particles through oncolysis, which then infect and kill neighboring tumor cells. Oncolytic viruses may induce therapeutic anti-cancer effects by selective targeting against cancer cells, thus, marking the malignant cells as targets for immunotherapy by the immune system that recognizes foreign antigens, thus, stimulating the host anti-tumor immune system responses. The therapy approach using oncolytic viruses has been accepted as a standard therapy since Imlygic® was approved by the Food and Drug Administration and European Medicines Agency for melanoma treatment in 2015. Oncolytic viruses are currently undergoing clinical trials against melanoma, glioma, pancreatic, and breast cancers. Oncolytic viruses with potential as therapeutics for human use are based on adenoviruses, Herpes Simplex Viruses, Vaccinia viruses, Reoviruses, Measle virus, Maraba virus, Myxoma virus, Newcastle disease virus, Parvovirus, viruses of the Paramyxoviridae family, Picornaviruses, and retroviral replicating vectors.

However, there is still an unmet need for new therapeutic options. In particular, there is still an unmet need for effective and inexpensive, easy to administer, transport and storage stable and safe cancer therapeutics.

The potential of viruses of the Birnaviridae family as cancer therapeutics for human use has not been discussed in the art yet. Birnaviridae are small (70 nm in diameter), non-enveloped viruses, with segmented, linear double stranded (dsRNA) genome which codes for 5 to 6 proteins positioned on 2 genome segments A and B. Birds, fishes, and insects serve as natural hosts. Infectious bursal disease virus (IBDV) is currently the only species within the Avibirnavirus genus. IBDV is a common poultry pathogen causing the infectious bursal disease (IBD), which is also known as Gumboro disease. It infects B cell precursors in young chickens. Vaccine strains are safely used in mass vaccinations for 50+ years. Birnaviruses typically release their progeny via continuous budding of viral particles from an intact cell membrane. This makes efficient oncolysis less likely.

A review by Qin and Zheng (Int. J. Mol. Sci. 2017, 18, 161; doi: 10.3390/ijms18010161) describes apoptosis and immunosuppression induced by IBDV proteins via interaction with cellular targets in avian cells. The capsid protein VP2 and the non-structural protein VP5 induce the programed cell death process via interacting with oral cancer overexpressed 1 (ORAOV1) (VP2), voltage-dependent anion channel 2 (VDAC2) (VP5), and receptor of activated protein kinase C1 (RACK1) (VP5) in chicken DF-1 cells. VP4, the viral protease, suppresses type I interferon expression via binding to glucocorticoid-induced leucine zipper protein (GILZ) in DF-1 cells. VP3 binds dsRNA and may also contribute to the blockage of viral dsRNA interacting to melanoma differentiation-associated protein 5 (MDA5) that detects dsRNA in the cytoplasm and initiates the innate immune response. Viral mechanisms to use cellular pathways or directed against the host's antiviral response evolve in a coevolution and often determine host range/species specificity. However, ORAOV1 and RACK1 are highly conserved eukaryotic proteins. As GILZ is far less conserved, the suppression of interferon induction by proteolytic activity of VP4 could be specific for avian host GILZ. Furthermore, VP2 expression triggers activation of double-stranded RNA-dependent protein kinase (PKR) and, as a result, eIF2a phosphorylation, which causes inhibition of viral gene translation and apoptosis. It is in a temporary balance with viral VP3 which inhibit this phosphorylation event and delays apoptosis. In this respect, the present inventors assume that in cells with RAS mutations, that also prevent PKR phosphorylation, virus can replicate more efficiently until VP2 causes apoptosis in cells with high virus load. Such mutations occur in approximately 30% of all human cancers making them to suitable targets for IBDV anticancer therapy.

The present inventors surprisingly found that viruses of the Birnaviridae family can be used as medicament, specifically as medicament in the treatment and/or prevention of cancer. In particular, the present inventors surprisingly found that viruses of the Birnaviridae family promote immunogenic cell death and immunomodulation and, thus, allow an efficient treatment of a broad range of different cancer types despite the fact that virus infection does not cause efficient cell lysis. By using the clinically proven attenuated live avian virus IBDV R903/78 for treatment of cancer in human, the present inventors surprisingly found that a pharmaceutical composition containing at least 10⁶ infectious units, preferably at least 10⁷ infectious units, more preferably at least 10⁸ infectious units, and even more preferably at least 10⁹ infectious units per dose is safe for humans. They further surprisingly found that a pharmaceutical composition comprising attenuated live avian virus IBDV R903/78 is suitable for the treatment of a large range of tumors including, but not limited to glioma including glioblastoma and advanced astrocytoma, advanced head and neck cancers, cholangiocarcinoma and multiple myeloma. These findings show that viruses of the Birnaviridae family combine the ability to kill tumor cells (e.g. via induction of apoptosis via triggering RACK1, DVAC2 and ORAOV1 protein degradation and via PKR phosphorylation) and properties for passive cancer immunotherapy (e.g. via interferon induction initiated by the dsRNA of an apathogenic virus). Interestingly, selective targeting of cancer cells in contrast to normal cells may be accomplished by deficient interferon-dependent anti-viral activation by cancer cells. Especially, a medicament comprising a virus of the Birnaviridae family is therapeutically efficient with oral application in tumors of the oral cavity and esophagus, but also in tumors not exposed to the upper gastrointestinal tract. According to the present inventors, the virus of the Birnaviridae family can be used in a monotherapy or in a combination therapy to treat or prevent cancer.

### SUMMARY OF THE INVENTION

In a first aspect, the present invention relates to a birnavirus for use in the treatment or prevention of cancer.

In a second aspect, the present invention relates to a combination comprising at least one birnavirus and at least one further active agent for use in the treatment or prevention of cancer.

In a third aspect, the present invention relates to a pharmaceutical composition comprising a birnavirus as defined in the first aspect or a combination as defined in the second aspect for use in the treatment or prevention of cancer.

In a fourth aspect, the present invention relates to a reservoir comprising a birnavirus as defined in the first aspect, a combination as defined in the second aspect, or a pharmaceutical composition as defined in the third aspect, wherein the reservoir is designed to be inserted into an oral or a nasal applicator.

In a fifth aspect, the present invention relates to an oral or nasal applicator comprising
(i) a reservoir comprising a birnavirus as defined in the first aspect, a combination as defined in the second aspect, or a pharmaceutical composition as defined in the third aspect, and
(ii) a mouth piece or nose piece, wherein the mouth piece or the nose piece is connected with/attached to the reservoir.

In a sixth aspect, the present invention relates to a kit comprising
(i) a packaging material, and
(ii) a birnavirus as defined in the first aspect, a combination as defined in the second aspect, a pharmaceutical composition as defined in the third aspect, a reservoir of the fourth aspect, and/or an oral or nasal applicator of the fifth aspect.

In a seventh aspect, the present invention relates to a birnavirus for use as medicament, wherein the birnavirus is to be administered to a subject at a dose of at least 10⁶ infectious units per day, preferably of at least 10⁷ infectious units per day, more preferably of at least 10⁸ infectious units per day, and even more preferably of at least 10⁹ infectious units per day.

In an eighth aspect, the present invention relates to a birnavirus for use as medicament, wherein the birnavirus is to be administered to a subject orally, nasally, or by inhalation.

In a ninth aspect, the present invention relates to a birnavirus for use in the treatment of subjects suffering from cancer, wherein the subjects are characterized as suffering from a cancer overexpressing oral cancer overexpressed 1 (ORAOV1), voltage-dependent anion channel 2 (VDAC2), and/or receptor of activated protein kinase C1 (RACK1), a cancer comprising cancer cells carrying a RAS mutation, and/or a cancer characterized by decreased or inhibited phosphorylation of RNA (dsRNA)-dependent protein kinase (PKR) activity.

In a tenth aspect, the present invention relates to a pharmaceutical composition comprising a birnavirus as defined in the first aspect or a combination as defined in the second aspect, wherein the composition is in a form suitable for oral administration, nasal administration, or administration by inhalation.

This summary of the invention does not describe all features of the invention.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. As used in this specification and the appended claims, the singular forms “a”, “an”, and “the” include plural referents, unless the content clearly dictates otherwise.

The term “consisting essentially of”, as used herein, limits the scope of a claim to the specified materials or steps and those that do not materially affect the basic and novel characteristic(s) of the claimed invention. In other words, the term “consisting essentially of”, as used herein, is generally construed to mean that the composition or formulation (a) necessarily includes the listed ingredients and (b) is open to unlisted ingredients that do not materially affect the basic and novel properties of the composition. Similarly, when “consisting essentially of” is used herein in a process claim, the claim requires that the listed steps are performed, but also may include unlisted steps that do not affect the basic and material properties of the process.

The term “about” when used in connection with a numerical value is meant to encompass numerical values within a range having a lower limit that is 5% smaller than the indicated numerical value and having an upper limit that is 5% larger than the indicated numerical value.

The term “oncolytic virus,” as used herein, refers to a virus that preferentially targets and/or kills cancer cells through infection. The oncolytic virus multiplies in infected cancer cells and releases new infectious virus particles through oncolysis, which then infect and kill neighboring tumor cells. Oncolytic viruses are thought not only to cause direct destruction of the tumor cells, but also to stimulate the host anti-tumor immune system response. The birnavirus described herein is an oncolytic virus.

The term “birnavirus”, as used herein, refers to a small (about 70 nm in diameter), non-enveloped virus. It is a segmented, linear, double-stranded (ds) RNA virus. The genome is about 5.9 to 6.9 kbp in length and codes for 5 to 6 proteins in segments A (Genebank Accession Number: JQ411012.1) and B (Genebank Accession Number: JQ411013.1). Birds, fishes, and insects are described as natural hosts. The birnavirus replication is cytoplasmic. Entry into the host cell is achieved by cell receptor endocytosis. Replication follows the double-stranded RNA virus replication model in the cytoplasm. Double-stranded RNA virus transcription is the method of transcription in cytoplasm. A birnavirus typically releases its progeny via continuous budding of viral particles from an intact cell membrane.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the (genome of) IBDV of strain 903/78 comprises a nucleotide sequence according to SEQ ID NO: 1 (segment A), a fragment thereof or a sequence having at least 80% sequence identity thereto, and/or
a nucleotide sequence according to SEQ ID NO: 2 (segment B), a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), preferably a nucleotide sequence that is a fragment which is between 1 and 12, more preferably between 1 and 8, and most preferably between 1 and 5 or 1 and 3, i.e. 1, 2, 3, 4, 5, 6, 7, 8, 9, 10, 11, or 12, nucleotides shorter than the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

The similarity of nucleotide sequences, i.e. the percentage of sequence identity, can be determined via sequence alignments. Such alignments can be carried out with several art-known algorithms, preferably with the mathematical algorithm of Karlin and Altschul (Karlin & Altschul (1993) Proc. Natl. Acad. Sci. USA 90:5873-5877), with hmmalign (HMMER package, http://hmmer.wustl.edu/) or with the CLUSTAL algorithm (Thompson J. D. et al. Nucleic Acids Res. 1994, 22:4673-80) available e.g. on http://www.ebi.ac.uk/Tools/clustalw/or on http://www.ebi.ac.uk/Tools/clustalw2/index.html or on http://npsa-pbil.ibcp.fr/cgi-bin/npsa_automat.pl?page=/NPSA/npsa_clustalw.html. Preferred parameters used are the default parameters as they are set on http://www.ebi.ac.uk/Tools/clustalw/ or http://www.ebi.ac.uk/Tools/clustalw2/index.html. The grade of sequence identity (sequence matching) may be calculated using e.g. BLAST, BLAT or BlastZ (or BlastX). A similar algorithm is incorporated into the BLASTN and BLASTP programs of Altschul et al. J. Mol. Biol. 1990, 215:403-410. To obtain gapped alignments for comparative purposes, Gapped BLAST is utilized as described in Altschul et al. Nucleic Acids Res. 1997, 25:3389-3402. When utilizing BLAST and Gapped BLAST programs, the default parameters of the respective programs are used. Sequence matching analysis may be supplemented by established homology mapping techniques like Shuffle-LAGAN (Brudno M., Bioinformatics 2003b, 19 Suppl 1:154-162) or Markov random fields.

The birnavirus of the present invention is apathogenic (i.e. not capable of causing disease) in humans and in the respective natural host.

The term “live virus” as used herein, refers to a virus that is capable of multiplying and producing progeny virus upon infection of a permissive isolated cell or a permissive cell as part of an organism. Such cell may be permissive by nature or acquire permissiveness via introduction of functional sequences required for replication or mutation/deletion of sequences that would otherwise prevent multiplication.

The term “attenuated virus”, as used herein, refers to a virus with compromised virulence in the intended recipient, e.g. subject as defined herein. The birnavirus described herein is preferably a live virus and more preferably a live and attenuated virus. In particular, the birnavirus described herein is a replication competent live and attenuated virus.

The term “treatment”, in particular “therapeutic treatment”, as used herein, refers to any therapy which improves the health status and/or prolongs (increases) the lifespan of a subject suffering from a disease such as cancer. Said therapy may eliminate the disease in a subject, arrest or slow the development of the disease in a subject, inhibit the development of the disease in a subject, decrease the severity of symptoms in a subject suffering the disease, and/or decrease the recurrence in a subject who currently has or who previously has had a disease.

The present invention relates to the use of a birnavirus as medicament. In particular, the present invention relates to the use of a birnavirus in the treatment or prevention of cancer.

The term “treatment of cancer”, as used herein, means accomplishing one or more of the following: (i) tumor growth inhibition and/or tumor cell death, (ii) reduction of tumor marker(s), (iii) reduction of tumor lesions and metastases, (iv) reduction of tumor burden as evidenced by imaging studies (e.g. CT, MRI, PET etc.), and (v) reduction of tumor burden as evidenced by clinical appraisal or self-report by the subject.

The term “prevention of cancer”, as used herein, means preventing any symptoms of cancer from occurring in a subject. For example, a prophylactic administration of a birnavirus of the present invention can protect the receiving subject from developing cancer. One aspect for cancer prevention may be accomplished by prevention of progression of cancer in patients at high risk to develop cancer, where cancer initiating cells may already be on board (e.g. patients with high risk cancer in remission but with high likelihood that “the last cancer initiating cells” still exist, e.g. patients with virus (HBV, HCV) induced liver cirrhosis that may progress to hepatocellular carcinoma (HCC), patients with cervical intraepithelial neoplasia that may progress to cervix cancer, patients with HIV-1 infection, patients with genetic susceptibility to develop cancer, or organ allograft recipients with life-long immunosuppressive treatment, etc.). Another aspect for cancer prevention may be accomplished by type 1 interferon secretion by normal cells in patients at risk of recurrent disease or for development of cancer, as listed above.

The term “cancer”, as used herein, refers to a group of diseases involving abnormal cell growth with the potential to invade or spread to other parts of the body. These contrast with benign tumors, which do not spread. The term “cancer” according to the invention also comprises cancer metastases.

In particular, the cancer to be treated can be in very early stages, from pre-malignant lesions, through primary tumors (non-metastatic) to metastatic stages (primary tumors with lymph node involvement, vessel invasion, lymph vessel invasion, distant organ metastases of all sites). In one embodiment, cancer only appears as primary tumor (non-metastatic). In one further embodiment, cancer only appears as metastasis without a designated primary tumor (cancer of unknown primary). In one another embodiment, the metastatic primary tumor has/had been removed surgically and an adjuvant treatment is needed to treat (suspected) microscopic remaining tumor burden, which then is performed in form of combination treatment with a birnavirus.

The cancer, as described herein, is preferably characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity.

Specifically, the term “cancer”, as used herein, includes, but is not limited to, lung cancer, colorectal cancer, head and neck cancer, stomach cancer, urothelial cancer, breast cancer, cervical cancer, endometrial cancer, ovarian cancer, melanoma (skin cancer), pancreatic cancer, brain cancer, prostate cancer, thyroid cancer, renal cancer, adrenal cancer, liver cancer especially hepatocellular carcinoma, lymphoma (cancer of the lymphocytes), or leukaemia (blood cancer).

In one preferred embodiment, the cancer is selected from the group consisting of a solid cancer, a cancer affecting the hematopoietic system, and a melanoma. In one more preferred embodiment, the solid cancer is a carcinoma or sarcoma. In one even more preferred embodiment, the carcinoma is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and hepatocellular carcinoma. In one alternative even more preferred embodiment, the sarcoma is selected from the group consisting of a sarcoma arising in bone, muscle, fat, blood vessels, cartilage, and other soft or connective tissue of the body. In one alternative more preferred embodiment, the cancer affecting the hematopoietic system is a lymphoma or leukemia. In a specific embodiment, the carcinoma is not liver cancer, especially hepatocellular carcinoma. The above-mentioned specific cancer types may further be characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity.

The term “monotherapy”, as used herein, refers to any therapy with only one active substance. In the context of the present invention, the active substance used in monotherapy to treat or prevent cancer is a birnavirus. The term “combination therapy”, as used herein, refers to any therapy with two or more active substances. In the context of the present invention, the active substances used in combination therapy to treat or prevent cancer are at least one birnavirus and at least one further active agent. The advantage of monotherapy is the possibility of a more targeted treatment of a disease, whereas combination therapy is intended to have a broader effect and also to eliminate side effects.

The term “active agent” as used herein, refers to any agent allowing the treatment, amelioration, and/or prevention of cancer. In addition, the term “active agent”, as used herein, refers to any therapeutic and/or preventive activity an agent may exhibit. The active agent is preferably selected from the group consisting of an immunomodulatory agent, a chemotherapeutic agent, an immunotherapeutic agent, an immunosuppressive agent, and an antibody.

The birnavirus may be administered to a subject receiving or having received at least one further anti-cancer therapy. The at least one further anti-cancer therapy may be selected from the group consisting of immunomodulatory therapy, chemotherapy, immunotherapy, radiation therapy, vaccination, stem cell therapy, anti-hormonal therapy, immunosuppressive therapy, antibody therapy, and surgery.

The term “immunomodulatory therapy”, as used herein, refers to an approach of treating cancer by stimulating T-cell function. In particular, immune modulation is based on the striking finding that stimulation of T-cell function with compounds that block or activate regulatory receptors, e.g. antibodies, is sufficient to cause the regression of some tumors. For example, immunomodulatory monoclonal antibodies (mAbs) target immune cells rather than cancer cells, and thus, are not necessarily specific to any cancer type.

Checkpoint blockade is, for example, a method by which T-cell function is stimulated with compounds that block their inhibitory receptors, whereas T-cell co-stimulation is a method that aims at activating T-cell function with compounds that target their stimulatory receptors. The immunomodulatory therapy described herein preferably encompasses the administration of a checkpoint inhibitor, more preferably a checkpoint inhibitor targeting programmed cell-death protein 1 (PD-1), programmed cell-death ligand 1 (PD-L1), programmed cell-death ligand 2 (PD-L2), cytotoxic T-lymphocyte-associated Protein 4 (CTLA-4) or intrinsic checkpoint blockades. In particular, the checkpoint inhibitor is an antibody such as a mAbs.

The term “chemotherapy”, as used herein, relates to a type of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). The chemotherapy described herein preferably encompasses the administration of an alkylating agent, an antimetabolite, folinic acid, a folate antagonist, a mitotic inhibitor, an anthracyclin, a topoisomerase inhibitor, an antibody, a signal transduction inhibitor, an inhibitor of angiogenesis, and/or an inhibitor of histone deacetylase.

The term “immunotherapy”, as used herein, refers to an approach of treating cancer by generating or augmenting an immune response against it. Immunotherapies designed to elicit or amplify an immune response are classified as activation immunotherapies, while immunotherapies that reduce or suppress an immune response are classified as suppression immunotherapies. An antitumor immunotherapy has broad potential and can be used to treat many different types of advanced-stage cancer owing to the durable and robust responses it elicits across a diverse spectrum of malignancies. Two types of immunotherapy have emerged as particularly effective over the past decade: immune-cell-targeted monoclonal antibody (mAb) therapy and adoptive cellular therapy (ACT). The immunotherapy described herein preferably encompasses the administration of a checkpoint inhibitor, a cytokine, an antibody, an antigen-presenting cell, and/or a chimeric antigen receptor T cell.

The term “radiation therapy”, as used herein, relates to a type of cancer treatment using ionizing radiation to control or kill cancerous/malignant cells. Ionizing radiation is normally delivered by a linear accelerator. Radiation therapy may be curative in a number of types of cancer if they are localized to one area of the body. It may also be used as part of adjuvant therapy, to prevent tumor recurrence after surgery to remove a primary malignant tumor (for example, early stages of breast cancer). Radiation therapy is synergistic with chemotherapy, and has been used before, during, and after chemotherapy in susceptible cancers.

The term “vaccination”, as used herein, describe the process of administering an antigen to a subject with the purpose of inducing an immune response, for example, for therapeutic or prophylactic reasons.

The term “antibody”, as used herein, refers to a glycoprotein comprising at least two heavy (H) chains and two light (L) chains inter-connected by disulfide bonds, and includes any molecule comprising an antigen binding portion thereof. The term “antibody” includes monoclonal antibodies and fragments or derivatives of antibodies, including, without limitation, human antibodies, humanized antibodies, chimeric antibodies, single chain antibodies, e.g., scFv's and antigen-binding antibody fragments such as Fab and Fab′ fragments and also includes all recombinant forms of antibodies, e.g., antibodies expressed in prokaryotes, unglycosylated antibodies, and any antigen-binding antibody fragments and derivatives as described herein. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen”, as used herein, relates to an agent such as a protein or peptide comprising an epitope against which an immune response is directed and/or is to be directed. The term “antigen presenting cell (APC)”, as used herein, is a cell of a variety of cells capable of displaying, acquiring, and/or presenting at least one antigen or antigenic fragment on (or at) its cell surface.

The terms “T cells” or “T lymphocytes”, as used herein, relate to types of lymphocytes that play a central role in cell-mediated immunity. T cells or T lymphocytes can be distinguished from other lymphocytes, such as B cells and natural killer (NK) cells by the presence of a T cell receptor (TCR) on the cell surface. They do not have antigen presenting properties (but rather, requiring B cells or NK cells for its antigen-presenting property). They are called T cells because they mature in the thymus. T cells are capable of recognizing an antigen when displayed on the surface of antigen presenting cells or matrix together with one or more MHC molecules or one or more non-classical MHC molecules.

As used herein, the expressions “is for administration” and “is to be administered” have the same meaning as “is prepared to be administered”. In other words, the statement that an active compound “is for administration” has to be understood in that said active compound has been formulated and made up into doses so that said active compound is in a state capable of exerting its therapeutic activity. In the context of the present invention, a birnavirus, a combination comprising at least one birnavirus and at least one further active agent, or a pharmaceutical composition comprising the birnavirus or the combination is prepared to be administered.

The birnavirus, the combination comprising at least one birnavirus and at least one further active agent, or the pharmaceutical composition comprising the birnavirus or the combination is preferably in a form suitable for oral administration, nasal administration, administration by inhalation, intravascular administration, intravenous administration, intramuscular administration, intrathecal administration, subcutaneous administration, or intraperitoneal administration. The birnavirus, the combination comprising at least one birnavirus and at least one further active agent, or the pharmaceutical composition comprising the birnavirus or the combination is more preferably in a form suitable for oral administration, nasal administration, or administration by inhalation.

The terms “therapeutically effective amount” or “therapeutic amount” are intended to mean that amount of a drug or pharmaceutical agent that will elicit the biological or medical response of a tissue, a system, animal or human that is being sought by a researcher, veterinarian, medical doctor or other clinician. The dosage regimen utilizing the birnavirus or the birnavirus in combination with the further active agent as described herein can be selected by the skilled practitioner in accordance with a variety of factors including type, species, age, weight, body mass index, sex and medical condition of the subject; the severity of the condition to be treated; the potency of the compound chosen to be administered; the route of administration; the purpose of the administration; and the renal and hepatic function of the subject. In the context of the present invention, the birnavirus, the combination comprising at least one birnavirus and at least one further active agent, or the pharmaceutical composition comprising the birnavirus or the combination is administered or prepared to be administered in a therapeutically effective/therapeutic amount.

The dose at which the birnavirus is to be administered preferably amounts to at least 10⁶ infectious units per day, more preferably to at least 10⁷ infectious units per day, even more preferably to at least 10⁸ infectious units per day, and most preferably to at least 10⁹ infectious units per day.

The pharmaceutical composition in accordance with the present invention may comprise one or more excipient(s), diluent(s), and/or carrier(s), all of which are preferably pharmaceutically acceptable. The term “pharmaceutically acceptable”, as used herein, means approved by a regulatory agency of the Federal or a state government or listed in the U.S. Pharmacopeia, European Pharmacopeia (Ph. Eur.) or other generally recognized pharmacopeia for use in animals, and more particularly in humans.

The term “excipient”, as used herein, is intended to indicate all substances in a pharmaceutical composition which are not active ingredients such as binders, lubricants, thickeners, surface active agents, preservatives, emulsifiers, buffers, flavoring agents, or colorants.

The term “diluent”, as used herein, relates to a diluting and/or thinning agent. Moreover, the term “diluent” includes a solution, suspension (e.g. liquid or solid suspension) and/or media.

The term “carrier”, as used herein, relates to one or more compatible solid or liquid fillers, which are suitable for an administration, e.g. to a human. The term “carrier” relates to a natural or synthetic organic or inorganic component which is combined with an active component in order to facilitate the application of the active component. Preferably, carrier components are sterile liquids such as water or oils, including those which are derived from mineral oil, animals, or plants, such as peanut oil, soy bean oil, sesame oil, sunflower oil, etc. Salt solutions and aqueous dextrose and glycerin solutions may also be used as aqueous carrier compounds.

Pharmaceutically acceptable carriers or diluents for therapeutic use are well known in the pharmaceutical art, and are described, for example, in Remington's Pharmaceutical Sciences, Mack Publishing Co. (A. R Gennaro edit. 1985). Examples of suitable carriers include, for example, magnesium carbonate, magnesium stearate, talc, lactose, pectin, dextrin, starch, gelatin, tragacanth, methylcellulose, sodium carboxymethylcellulose, a low melting wax, cocoa butter, and the like. Examples of suitable diluents include ethanol, glycerol, and water.

Pharmaceutical carriers, diluents, and/or excipients can be selected with regard to the intended route of administration and standard pharmaceutical practice. The pharmaceutical compositions of the present invention may comprise as, or in addition to, the carrier(s), excipient(s) or diluent(s) any suitable binder(s), lubricant(s), suspending agent(s), coating agent(s), and/or solubilising agent(s). Examples of suitable binders include starch, gelatin, natural sugars such as glucose, lactose, sucrose, trehalose, corn sweeteners, natural and synthetic gums, such as acacia, tragacanth or sodium alginate, carboxymethyl cellulose, and polyethylene glycol. Examples of suitable lubricants include sodium oleate, sodium stearate, magnesium stearate, sodium benzoate, sodium acetate, sodium chloride, and the like. Preservatives, stabilizers, dyes, and even flavoring agents may be provided in the pharmaceutical composition. Examples of preservatives include sodium benzoate, sorbic acid, and esters of p-hydroxybenzoic acid. Antioxidants and suspending agents may be also used.

The term “systemic administration”, a used herein, refers to the administration of the birnavirus, the combination comprising at least one birnavirus and at least one further active agent, or the pharmaceutical composition comprising the birnavirus or the combination such that said birnavirus, combination, or pharmaceutical composition becomes widely distributed in the body of a subject in significant amounts and develops a biological effect. Typical systemic routes of administration include administration by introducing the birnavirus, the combination comprising at least one birnavirus and at least one further active agent, or the pharmaceutical composition comprising the birnavirus or the combination directly into the vascular system, wherein said birnavirus, combination, or pharmaceutical composition enters the vascular system and is carried to one or more desired site(s) of action via the blood. The systemic administration may be by parenteral administration. The term “parenteral administration”, as used herein, refers to the administration of the birnavirus, the combination comprising at least one birnavirus and at least one further active agent, or the pharmaceutical composition comprising the birnavirus or the combination such that said birnavirus, combination, or pharmaceutical composition does not pass the intestine. The term “parenteral administration” includes intravenous administration, subcutaneous administration, intradermal administration, or intraarterial administration, but is not limited thereto. The birnavirus, the combination comprising at least one birnavirus and at least one further active agent, or a pharmaceutical composition comprising the birnavirus or the combination is preferably administered orally, nasally, or by inhalation.

The term “subject”, as used herein, refers to any individual which may receive a birnavirus, a combination comprising at least one birnavirus and at least one further active agent, or a pharmaceutical composition comprising the birnavirus or the combination of the present invention. The term “subject”, as used herein, refers to any individual that/who may benefit from the treatment with a birnavirus, a combination comprising at least one birnavirus and at least one further active agent, or a pharmaceutical composition comprising the birnavirus or the combination of the present invention. The subject is prevented for getting cancer or treated for cancer.

The subject is preferably a subject suffering from (i) a cancer overexpressing oral cancer overexpressed 1 (ORAOV1), voltage-dependent anion channel 2 (VDAC2), and/or receptor of activated protein kinase C1 (RACK1), (ii) a cancer characterized by cancer cells carrying a RAS mutation, and/or (iii) a cancer characterized by decreased or inhibited phosphorylation of RNA (dsRNA)-dependent protein kinase (PKR) activity. A population of subjects (patients) suffering from cancer having the above characteristics can be treated with a birnavirus, combination, or composition of the present invention.

The subject may be a vertebrate, e.g. a human being, dog, cat, sheep, goat, cow, horse, camel, or pig. It is particularly preferred that the “subject” is a human being.

The terms “subject”, “individual”, or “patient” are used interchangeably herein.

In the context of the present invention, the term “kit of parts (in short: kit)” is understood to be any combination of at least some of the components identified herein, which are combined, coexisting spatially, to a functional unit, and which can contain further components.

### Embodiments of the Invention

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

The present inventors surprisingly found that viruses of the Birnaviridae family can be used as medicament. Specifically, the present inventors surprisingly found that viruses of the Birnaviridae family can be used for the treatment and/or prevention of cancer. In particular, the present inventors surprisingly found that viruses of the Birnaviridae family promote immunogenic cell death and immunomodulation and, thus, allow an efficient treatment of a broad range of different cancer types.

Thus, in a first aspect, the present invention relates to a birnavirus for use in the treatment or prevention of cancer.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1 (segment A), a fragment thereof or a sequence having at least 80% sequence identity thereto, and/or
a nucleotide sequence according to SEQ ID NO: 2 (segment B), a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The naturally or non-naturally occurring birnavirus may be rescued from a bacterial plasmid designed for expression of birnavirus RNAs. The non-naturally occurring birnavirus is preferably a mutated birnavirus or a chemically treated birnavirus (e.g. treated with a protease).

In one preferred embodiment, the cancer is selected from the group consisting of a solid cancer, a cancer affecting the hematopoietic system, and a melanoma. In one more preferred embodiment, the solid cancer is a carcinoma or sarcoma. In one alternative more preferred embodiment, the cancer affecting the hematopoietic system is a lymphoma or leukemia. In one even more preferred embodiment, the carcinoma is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and liver carcinoma. In one alternative even more preferred embodiment, the sarcoma is selected from the group consisting of a sarcoma arising in bone, muscle, fat, blood vessels, cartilage, and other soft or connective tissue of the body. In a specific embodiment, the carcinoma is no liver carcinoma.

Alternatively or additionally, the cancer is characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity.

In one preferred embodiment, the birnavirus is for use in the treatment or prevention of cancer in a subject, wherein the subject suffers from (i) a cancer overexpressing oral cancer overexpressed 1 (ORAOV1), voltage-dependent anion channel 2 (VDAC2), and/or receptor of activated protein kinase C1 (RACK1), (ii) a cancer characterized by cancer cells carrying a RAS mutation, and/or (iii) a cancer characterized by decreased or inhibited phosphorylation of RNA (dsRNA)-dependent protein kinase (PKR) activity.

The dose at which the birnavirus is to be administered preferably amounts to at least 10⁶ infectious units per day, more preferably to at least 10⁷ infectious units per day, even more preferably to at least 10⁸ infectious units per day, and most preferably to at least 10⁹ infectious units per day.

In one particularly preferred embodiment, the birnavirus is for use in the treatment or prevention of cancer, wherein the birnavirus is an Infectious Bursal Disease Virus (IBDV) and wherein the cancer is characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity. The dose at which the IBDV is to be administered preferably amounts to at least 10⁶ infectious units per day, more preferably to at least 10⁷ infectious units per day, even more preferably to at least 10⁸ infectious units per day, and most preferably to at least 10⁹ infectious units per day.

In one particularly preferred embodiment, the birnavirus is for use in the treatment or prevention of cancer, wherein the birnavirus is an Infectious Bursal Disease Virus (IBDV), and wherein the cancer is selected from the group consisting of a breast cancer, head and neck cancer, colorectal cancer, pancreatic cancer, ovarian cancer, liver cancer, a glioblastoma, a lymphoma, adrenal cancer, lung cancer, prostate cancer, thyroid cancer, renal cancer and leukemia. The dose at which the IBDV is to be administered preferably amounts to at least 10⁶ infectious units per day, more preferably to at least 10⁷ infectious units per day, even more preferably to at least 10⁸ infectious units per day, and most preferably to at least 10⁹ infectious units per day.

By using the clinically proven attenuated live avian virus IBDV R903/78 for the treatment or prevention of cancer in humans, the present inventors surprisingly found that a dose of at least 10⁶ infectious units per day, preferably of at least 10⁷ infectious units per day, more preferably of at least 10⁸ infectious units per day, and even more preferably of at least 10⁹ infectious units per day is safe for human beings.

In one preferred embodiment, the birnavirus is for administration to a subject receiving or having received at least one further anti-cancer therapy. In this regard, the administration of the birnavirus is considered to be a first anti-cancer therapy and the further anti-cancer therapy is considered to be a second anti-cancer therapy.

In one more preferred embodiment, the at least one further anti-cancer therapy is selected from the group consisting of immunomodulatory therapy, chemotherapy, immunotherapy, radiation therapy, vaccination, stem cell therapy, anti-hormonal therapy, immunosuppressive therapy, antibody therapy, and surgery.

The different anti-cancer therapies can also be combined with each other. It is particularly preferred that the birnavirus is for administration to a subject receiving (i) an immunomodulatory therapy, (ii) a chemotherapy, (iii) an immunotherapy, (iv) an immunomodulatory therapy and a chemotherapy, (v) a chemotherapy and an immunotherapy, (vi) an immunomodulatory therapy and an immunotherapy, or (vii) an immunomodulatory therapy, a chemotherapy, and an immunotherapy.

In those embodiments, in which the anti-cancer-therapy is an immunomodulatory therapy, said immunomodulatory therapy includes all interventions aimed at modulating (activating or inhibiting) specific elements of the immune system of the host or the tumor microenvironment (e.g. CTLA-4 blockade, anti-PD-L1 therapy, macrophage modulation, CD40L agonists etc.).

In those embodiments, in which the anti-cancer-therapy is chemotherapy, said chemotherapy includes the administration of a chemotherapeutic agent.

In those embodiments, in which the anti-cancer-therapy is an immunotherapy, said immunotherapy includes all interventions using immunological mechanisms (i.e. adoptive T cell transfer, chimeric antigen receptor therapy, antibodies against tumor targets, anti-PD-1 therapy, etc.).

In those embodiments, in which the anti-cancer-therapy is radiation therapy, said radiation therapy may include all forms of radiation, including heavy ion. The radiation therapy as further anti-cancer therapy does not necessarily need to be within a given therapeutic intensity, single doses as low as 1 Gy can have beneficial effects in combination with a birnavirus. A single dose may have a low or high intensity. A low intensity is in the range of 1-30 Gy, i.e. 1, 2, 3, 4, 5, 6, 7, 8, 9, 10, 15, 20, 25, 26, 27, 28, 29, or 30 Gy. A high intensity is within the range of 31-100 Gy, i.e. 31, 32, 33, 34, 35, 36, 37, 38, 39, 40, 45, 50, 55, 60, 65, 70, 75, 80, 85, 90, 95, or 100 Gy. Accordingly, a single dose may have an intensity of 1, 2, 3, 4, 5, 6, 7, 8, 9, 10, 15, 20, 25, 30, 35, 40, 45, 50, 55, 60, 65, 70, 75, 80, 85, 90, 95, or 100 Gy. The intensity of a single dose may, thus, be in the range of 1-80 Gy, 2-80 Gy, 3-80 Gy, 4-80 Gy, 5-80 Gy, 6-80 Gy, 7-80 Gy, 8-80 Gy, 9-80 Gy, 10-80 Gy, 15-80 Gy, 20-80 Gy, 25-80 Gy, 30-80 Gy, 35-80 Gy, 40-80 Gy, 45-80 Gy, 50-80 Gy, 55-80 Gy, 60-80 Gy, 65-80 Gy, 70-80 Gy, or 75-80 Gy.

In those embodiments, in which the anti-cancer-therapy is stem cell therapy, said stem cell therapy can be a treatment with interferon alpha or treatment with neutralizing antibodies against stem cell (growth) factors.

In those embodiments, in which the anti-cancer-therapy is anti-hormonal therapy, said anti-hormonal therapy may comprise androgen deprivation (anti-androgens or GnRH-antagonists or androgen-synthesis inhibitors or androgen uptake inhibitors), estrogen-deprivation (conversion inhibitors, aromatase inhibition, E2 blockade), progesterone-deprivation and others (e.g. abarelix, abriateronacetate, anastrozole, bicalutamid, buserelin, cyproteronacetate, degarelix, exemestan, flutamid, faslodex, goserelin, histrel, letrozole, leuprorelin, medroxyprogesterone, megestrol, tamoxifen, toremifen, triptorelin and others).

In one even more preferred embodiment,
(i) the immunomodulatory therapy encompasses the administration of a checkpoint inhibitor, preferably a checkpoint inhibitor targeting PD-1, PD-L1, PD-L2, CTLA-4 or intrinsic checkpoint blockades,
(ii) the chemotherapy encompasses the administration of an alkylating agent, an antimetabolite, folinic acid, a folate antagonist, a mitotic inhibitor, an anthracyclin, a topoisomerase inhibitor, an antibody, a signal transduction inhibitor, an inhibitor of angiogenesis, or an inhibitor of histone deacetylase, and/or
(iii) the immunotherapy encompasses the administration of a cytokine, an antibody, an antigen-presenting cell, or a chimeric antigen receptor T cell.

The alkylating agent can be selected from the group consisting of bendamustin, busulfan, carboplatin, carmustin, cisplatin, oxaliplatin, cyclophosphamid, mitomycin, and treosulfan.

The antimetabolite can be selected from the group consisting of 5-fluorouracil, capecitabin, cytarabin, gemcitabin, mercaptopurin, and deoxyglucose.

The folate antagonist can be selected from the group consisting of methotrexate and pemetrexed.

The mitotic inhibitor can be selected from the group consisting of taxanes and vinca-alcaloids. Taxanes suitable for use in the present invention include for example cabzitaxel, docetaxel, or paclitaxel. Paclitaxel can be administered as nanoparticle albumin bound paclitaxel (nab-paclitaxel, commercially available from Celgene Corp. under the trade name Abraxane).

The anthracyclin can be selected from the group consisting of bleomycin, doxorubicin, mitoxantron, and epirubicin.

The topoisomerase inhibitor can be selected from the group consisting of campthotecin derivatives and podophyllin derivatives. Campthotecin-derivatives suitable for use in the present invention include for example irinotecan and topotecan.

The cytokine can be selected from the group consisting of IL-2, IL7, IL-12 IL-15, IL-21 and GM-SCF.

The antibody can be selected from the group consisting of bevacizumab (Avastin®, Roche), cetuximab (Erbitux®, Bristol-Myers Squibb and Merck KGaA), panitumumab (Vectibix®, Amgen), ipilimumab, nuvolimumab, tremelimumab, anti-OX40-antibodies, catumaxomab, and anti-CD40L antibodies.

The signal transduction inhibitor can be selected from the group consisting of axatinib, crizotinib, erlotinib, sunitinib, sorafenib, everolimus, imatinib, lapatinib, pazopanib, temsirolimus, and vemurafenib.

An inhibitor of angiogenesis suitable for use in the present invention is, for example, aflibercept.

An inhibitor of histone deacetylase suitable for use in the present invention is, for example, vorinostat.

The birnavirus is for use in the treatment or prevention of cancer in a subject. The subject suffers from cancer or is at risk of developing cancer. The subject may be a mammal selected from the group consisting of a human being, dog, cat, sheep, goat, cow, horse, camel, and pig. It is particularly preferred that the mammal is a human being.

The first aspect of the present invention can alternatively be worded as follows: A method for preventing or treating cancer comprising the step of: administering a birnavirus to a subject (in need thereof), thereby preventing or treating cancer in the subject.

It is preferred that the birnavirus is administered in a therapeutically effective amount. It is (alternatively or additionally) further preferred that the subject is treated with the birnavirus prior to, during and/or after the subject was subjected to at least one further anti-cancer therapy. It is more preferred that the at least one further anti-cancer therapy is selected from the group consisting of immunomodulatory therapy, chemotherapy, immunotherapy, radiation therapy, vaccination, stem cell therapy, anti-hormonal therapy, immunosuppressive therapy, antibody therapy, and surgery. With regard to the preferred embodiments, reference is made to the above explanations.

The first aspect of the present invention can further alternatively be worded as follows: Use of a birnavirus for the preparation of a medicament for the treatment or prevention of cancer. With regard to the preferred embodiments, reference is made to the above explanations.

In a second aspect, the present invention relates to a combination comprising at least one birnavirus and at least one further active agent for use in the treatment or prevention of cancer. Thus, the at least one birnavirus and the at least one further active agent are used in a combination therapy.

The at least one birnavirus and the at least one further active agent may be present in the combination individually or together. For example, the at least one birnavirus may be comprised in a (first) composition and the at least one further active agent may be comprised in another/different (second) composition. Alternatively, the at least one birnavirus and the at least one further active agent may be comprised in a single composition.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

In one preferred embodiment, the cancer is selected from the group consisting of a solid cancer, a cancer affecting the hematopoietic system, and a melanoma. In one more preferred embodiment, the solid cancer is a carcinoma or sarcoma. In one alternative more preferred embodiment, the cancer affecting the hematopoietic system is a lymphoma or leukemia. In one even more preferred embodiment, the carcinoma is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and liver carcinoma. In one alternative even more preferred embodiment, the sarcoma is selected from the group consisting of a sarcoma arising in bone, muscle, fat, blood vessels, cartilage, and other soft or connective tissue of the body. In a specific embodiment, the carcinoma is no liver carcinoma.

Alternatively or additionally, the cancer is characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity.

In one preferred embodiment, the combination comprising at least one birnavirus and at least one further active agent is for use in the treatment or prevention of cancer in a subject, wherein the subject suffers from (i) a cancer overexpressing oral cancer overexpressed 1 (ORAOV1), voltage-dependent anion channel 2 (VDAC2), and/or receptor of activated protein kinase C1 (RACK1), (ii) a cancer characterized by cancer cells carrying a RAS mutation, and/or (iii) a cancer characterized by decreased or inhibited phosphorylation of RNA (dsRNA)-dependent protein kinase (PKR) activity.

The dose at which the birnavirus is to be administered preferably amounts to at least 10⁶ infectious units per day, more preferably to at least 10⁷ infectious units per day, even more preferably to at least 10⁸ infectious units per day, and most preferably to at least 10⁹ infectious units per day.

In one preferred embodiment, the combination comprising at least one birnavirus and at least one further active agent is for use in the treatment or prevention of cancer in a subject, wherein the at least one further active agent is selected from the group consisting of an immunomodulatory agent, a chemotherapeutic agent, an immunotherapeutic agent, an immunosuppressive agent, and an antibody.

In one more preferred embodiment,
(i) the immunomodulatory agent is selected from the group consisting of a checkpoint inhibitor, preferably a checkpoint inhibitor targeting PD-1, PD-L1, PD-L2, CTLA-4 or intrinsic checkpoint blockades,
(ii) the chemotherapeutic agent is selected from the group consisting of an alkylating agent, an antimetabolite, folinic acid, a folate antagonist, a mitotic inhibitor, an anthracyclin, a topoisomerase inhibitor, an antibody, a signal transduction inhibitor, an inhibitor of angiogenesis, and an inhibitor of histone deacetylase, and/or
(iii) immunotherapeutic agent is selected from the group consisting of a cytokine, an antibody, an antigen-presenting cell, or a chimeric antigen receptor T cell.

The alkylating agent can be selected from the group consisting of bendamustin, busulfan, carboplatin, carmustin, cisplatin, oxaliplatin, cyclophosphamid, mitomycin, and treosulfan.

The antimetabolite can be selected from the group consisting of 5-fluorouracil, capecitabin, cytarabin, gemcitabin, mercaptopurin, and deoxyglucose.

The folate antagonist can be selected from the group consisting of methotrexate and pemetrexed.

The mitotic inhibitor can be selected from the group consisting of taxanes and vinca-alcaloids. Taxanes suitable for use in the present invention include for example cabzitaxel, docetaxel, or paclitaxel. Paclitaxel can be administered as nanoparticle albumin bound paclitaxel (nab-paclitaxel, commercially available from Celgene Corp. under the trade name Abraxane).

The anthracyclin can be selected from the group consisting of bleomycin, doxorubicin, mitoxantron, and epirubicin.

The topoisomerase inhibitor can be selected from the group consisting of campthotecin derivatives and podophyllin derivatives. Campthotecin-derivatives suitable for use in the present invention include for example irinotecan and topotecan.

The cytokine can be selected from the group consisting of IL-2, IL7, IL-12 IL-15, IL-21 and GM-SCF.

The antibody can be selected from the group consisting of bevacizumab (Avastin®, Roche), cetuximab (Erbitux®, Bristol-Myers Squibb and Merck KGaA), panitumumab (Vectibix®, Amgen), ipilimumab, nuvolimumab, tremelimumab, anti-OX40-antibodies, catumaxomab, and anti-CD40L antibodies.

The signal transduction inhibitor can be selected from the group consisting of axatinib, crizotinib, erlotinib, sunitinib, sorafenib, everolimus, imatinib, lapatinib, pazopanib, temsirolimus, and vemurafenib.

An inhibitor of angiogenesis suitable for use in the present invention is, for example, aflibercept.

An inhibitor of histone deacetylase suitable for use in the present invention is, for example, vorinostat.

The at least one birnavirus and the at least one further active agent can be administered concurrently or consecutively. In those embodiments, in which the at least one birnavirus and the at least one further active agent are to be administered concurrently, said at least one birnavirus and said at least one further agent are preferably comprised in the combination together, e.g. in one single composition. In those embodiments, in which the at least one birnavirus and the at least one further active agent are to be administered consecutively, said at least one birnavirus and said at least one further agent are preferably comprised in the combination individually, e.g. the at least one birnavirus may be comprised in a (first) composition and the at least one further active agent may be comprised in another/different (second) composition.

It is particularly preferred that the combination comprises different birnaviruses (i.e. a mixture of birnaviruses). The different birnaviruses are preferably birnaviruses of different types or from different strains.

The combination is for use in the treatment or prevention of cancer in a subject. The subject suffers from cancer or is at risk of developing cancer. The subject may be a mammal selected from the group consisting of a human being, dog, cat, sheep, goat, cow, horse, camel, and pig. It is particularly preferred that the mammal is a human being.

The second aspect of the present invention can alternatively be worded as follows: A method for preventing or treating cancer comprising the step of: administering a combination comprising at least one birnavirus and at least one further active agent to a subject (in need thereof), thereby preventing or treating cancer in the subject.

It is preferred that the component(s) of the combination, i.e. the at least birnavirus and/or the at least one active agent, is (are) administered in a therapeutically effective amount. With regard to the preferred embodiments, reference is made to the above explanations.

The second aspect of the present invention can further alternatively be worded as follows: Use of a combination comprising at least one birnavirus and at least one further active agent for the preparation of a medicament for the treatment or prevention of cancer. With regard to the preferred embodiments, reference is made to the above explanations.

In a third aspect, the present invention relates to a pharmaceutical composition comprising a birnavirus as defined in the first aspect or a combination as defined in the second aspect for use in the treatment or prevention of cancer.

In one embodiment, the progression of cancer, when cancer cells exist, is prevented.

In one preferred embodiment, the pharmaceutical composition comprises one or more pharmaceutical acceptable excipient(s), diluent(s), and/or carrier(s).

The pharmaceutical composition can be administered systemically, e.g. parenterally. In one preferred embodiment, the pharmaceutical composition is in a form suitable for oral administration, nasal administration, or administration by inhalation. The pharmaceutical composition can also be administered intravascular, intravenous, intramuscular, intrathecal, subcutaneous, intraperitoneal, topical, rectal, vaginal, or by injection into or near the tumor.

The pharmaceutical composition can be administered in a single dose or in more than one dose. It is preferred that the pharmaceutical composition is to be administered in a therapeutically effective amount. The dose at which the pharmaceutical composition comprising a birnavirus as defined in the first aspect is to be administered preferably amounts to at least 10⁶ infectious units per day, more preferably to at least 10⁷ infectious units per day, even more preferably to at least 10⁸ infectious units per day, and most preferably to at least 10⁹ infectious units per day.

By using the clinically proven attenuated live avian virus IBDV R903/78 for the treatment or prevention of cancer in humans, the present inventors surprisingly found that a dose of at least 10⁶ infectious units per day, preferably of at least 10⁷ infectious units per day, more preferably of at least 10⁸ infectious units per day, and even more preferably of at least 10⁹ infectious units per day is safe for human beings. They further surprisingly found that a pharmaceutical composition comprising an IBDV is suitable for the treatment of a large range of tumors including, but not limited to, lung cancer, malignant gliomas, advanced head and neck cancers, and breast cancer. These findings show that viruses of the Birnaviridae family combine the ability to kill tumor cells (e.g. via induction of apoptosis via triggering RACK1, DVAC2 and ORAOV1 protein degradation and via PKR phosphorylation) and properties for passive cancer immunotherapy (e.g. via interferon induction initiated by the dsRNA of an apathogenic virus). Especially, the medicament comprising a virus of the Birnaviridae family is therapeutically efficient with oral application in tumors of the oral cavity and esophagus, but also in tumors not exposed to the upper gastrointestinal tract.

In one further preferred embodiment, the cancer is selected from the group consisting of a solid cancer, a cancer affecting the hematopoietic system, and a melanoma. In one more preferred embodiment, the solid cancer is a carcinoma or sarcoma. In one alternative more preferred embodiment, the cancer affecting the hematopoietic system is a lymphoma or leukemia. In one even more preferred embodiment, the carcinoma is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and liver carcinoma. In one alternative even more preferred embodiment, the sarcoma is selected from the group consisting of a sarcoma arising in bone, muscle, fat, blood vessels, cartilage, and other soft or connective tissue of the body. In a specific embodiment, the carcinoma is no liver carcinoma.

Alternatively or additionally, the cancer is characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity.

The pharmaceutical composition is for use in the treatment or prevention of cancer in a subject. The subject suffers from cancer or is at risk of developing cancer. The subject may be a mammal selected from the group consisting of a human being, dog, cat, sheep, goat, cow, horse, camel, and pig. It is particularly preferred that the mammal is a human being.

The third aspect of the present invention can alternatively be worded as follows: A method for preventing or treating cancer comprising the step of: administering a pharmaceutical composition comprising a birnavirus as defined in the first aspect or a combination as defined in the second aspect to a subject (in need thereof), thereby preventing or treating cancer in the subject.

With regard to the preferred embodiments, reference is made to the above explanations.

The third aspect of the present invention can further alternatively be worded as follows: Use of a pharmaceutical composition comprising a birnavirus as defined in the first aspect or a combination as defined in the second aspect for the preparation of a medicament for the treatment or prevention of cancer. With regard to the preferred embodiments, reference is made to the above explanations.

In a fourth aspect, the present invention relates to a reservoir comprising a birnavirus as defined in the first aspect, a combination as defined in the second aspect, or a pharmaceutical composition as defined in the third aspect, wherein the reservoir is designed to be inserted into an oral or a nasal applicator.

The reservoir may comprise or consist of one or more chambers for receiving a birnavirus as defined in the first aspect, a combination as defined in the second aspect, or a pharmaceutical composition as defined in the third aspect.

In those embodiments, in which only the birnavirus, the combination comprising the at least one birnavirus and the at least one further active agent together, or the pharmaceutical composition comprising the birnavirus or the at least one birnavirus and the at least one further active agent is to be administered, the reservoir comprises only one chamber. In those embodiments, in which the combination comprising the at least one birnavirus and the at least one further active agent individually is to be administered, the reservoir comprises two chambers, i.e. one chamber for the at least one birnavirus and one chamber for the at least one further active agent.

In a fifth aspect, the present invention relates to an oral or a nasal applicator comprising
(i) a reservoir comprising a birnavirus as defined in the first aspect, a combination as defined in the second aspect, or a pharmaceutical composition as defined in the third aspect, and
(ii) a mouth piece or nose piece, wherein the mouth piece or the nose piece is connected with/attached to the reservoir.

The oral or a nasal applicator allows the orally or nasally application of a birnavirus as defined in the first aspect, a combination as defined in the second aspect, or a pharmaceutical composition as defined in the third aspect.

The reservoir may comprise or consist of one or more chambers for receiving a birnavirus as defined in the first aspect, a combination as defined in the second aspect, or a pharmaceutical composition as defined in the third aspect.

In those embodiments, in which only the birnavirus, the combination comprising the at least one birnavirus and the at least one further active agent together, or the pharmaceutical composition comprising the birnavirus or the at least one birnavirus and the at least one further active agent is to be administered, the reservoir comprises only one chamber. In those embodiments, in which the combination comprising the at least one birnavirus and the at least one further active agent individually is to be administered, the reservoir comprises two chambers, i.e. one chamber for the at least one birnavirus and one chamber for the at least one further active agent.

In one preferred embodiment, the mouth piece or nose piece is designed so as to allow complete insertion into the mouth or nose.

In one further preferred embodiment, the oral or nasal applicator also comprises a discharge dosing unit for dosed release of a birnavirus as defined in the first aspect, a combination as defined in the second aspect, or a pharmaceutical composition as defined in the third aspect from the reservoir via the mouth or nose piece.

The oral or nasal applicator is configured to administer a dose of the birnavirus which preferably amounts to at least 10⁶ infectious units per day, more preferably to at least 10⁷ infectious units per day, even more preferably to at least 10⁸ infectious units per day, and most preferably to at least 10⁹ infectious units per day.

The oral or a nasal applicator is preferably used for the treatment or prevention of cancer in a subject. The subject suffers from cancer or is at risk of developing cancer. The subject may be a mammal selected from the group consisting of a human being, dog, cat, sheep, goat, cow, horse, camel, and pig. It is particularly preferred that the mammal is a human being.

In a sixth aspect, the present invention relates to a kit comprising
(i) a packaging material, and
(ii) a birnavirus as defined in the first aspect, a combination as defined in the second aspect, a pharmaceutical composition as defined in the third aspect, a reservoir of the fourth aspect, or an oral or nasal applicator of the fifth aspect.

The kit is preferably used for the treatment or prevention of cancer in a subject. The subject suffers from cancer or is at risk of developing cancer. The subject may be a mammal selected from the group consisting of a human being, dog, cat, sheep, goat, cow, horse, camel, and pig. It is particularly preferred that the mammal is a human being.

In one preferred embodiment, the kit further comprises
(iii) a label or packaging insert contained within the packaging material indicating that subjects
   receiving the birnavirus, the combination or the pharmaceutical composition can be prevented for getting cancer or treated for cancer, or
   using the reservoir or the oral or nasal applicator can be prevented for getting cancer or treated for cancer.

In one more preferred embodiment, the label or packaging insert further comprises the information that the dose at which the birnavirus is to be administered amounts to at least 10⁶ infectious units per day, more preferably to at least 10⁷ infectious units per day, even more preferably to at least 10⁸ infectious units per day, and most preferably to at least 10⁹ infectious units per day.

The birnavirus cannot only be used to prevent or treat cancer. It can generally also be used as a medicament, especially at a high dose of at least 10⁶ infectious units per day. The administration of a birnavirus at such a high dose has never been described before.

Thus, in a seventh aspect, the present invention relates to a birnavirus for use as medicament, wherein the birnavirus is to be administered to a subject (in need of said medicament) at a dose of at least 10⁶ infectious units per day, preferably of at least 10⁷ infectious units per day, more preferably of at least 10⁸ infectious units per day, and even more preferably of at least 10⁹ infectious units per day.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

In one further preferred embodiment, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

The subject may be a mammal selected from the group consisting of a human being, dog, cat, sheep, goat, cow, horse, camel, and pig. It is particularly preferred that the mammal is a human being.

As to the other preferred embodiments, it is referred to the first aspect of the present invention.

The present inventors surprisingly found that the birnavirus can easily and effectively be administered to a subject orally, nasally, or by inhalation. This has never been described before.

Thus, in an eight aspect, the present invention relates to a birnavirus for use as medicament, wherein the birnavirus is to be administered to a subject orally, nasally, or by inhalation.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

In one further preferred embodiment, the birnavirus is a live birnavirus. The birnavirus may be a naturally occurring or non-naturally occurring (live) birnavirus. The non-naturally occurring birnavirus is preferably a recombinant birnavirus. The non-naturally occurring birnavirus is preferably a mutated birnavirus or a chemically treated birnavirus (e.g. treated with a protease).

The subject may be a mammal selected from the group consisting of a human being, dog, cat, sheep, goat, cow, horse, camel, and pig. It is particularly preferred that the mammal is a human being.

As to the other preferred embodiments, it is referred to the first aspect of the present invention.

In a ninth aspect, the present invention relates to a birnavirus for use in the treatment of subjects suffering from cancer, wherein the subjects are characterized as suffering from
a cancer overexpressing oral cancer overexpressed 1 (ORAOV1), voltage-dependent anion channel 2 (VDAC2), and/or receptor of activated protein kinase C1 (RACK1),
a cancer comprising cancer cells carrying a RAS mutation, and/or
a cancer characterized by decreased or inhibited phosphorylation of RNA (dsRNA)-dependent protein kinase (PKR) activity.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or
a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i), and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

In one further preferred embodiment, the cancer is selected from the group consisting of a solid cancer, a cancer affecting the hematopoietic system, and a melanoma. In one more preferred embodiment, the solid cancer is a carcinoma or sarcoma. In one alternative more preferred embodiment, the cancer affecting the hematopoietic system is a lymphoma or leukemia. In one even more preferred embodiment, the carcinoma is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and liver carcinoma. In one alternative even more preferred embodiment, the sarcoma is selected from the group consisting of a sarcoma arising in bone, muscle, fat, blood vessels, cartilage, and other soft or connective tissue of the body. In a specific embodiment, the carcinoma is no liver carcinoma.

The subjects may be mammals selected from the group consisting of a human beings, dogs, cats, sheep, goats, cows, horses, and pigs. It is particularly preferred that the mammals are human beings.

As to the other preferred embodiments, it is referred to the first aspect of the present invention.

In a tenth aspect, the present invention relates to a pharmaceutical composition comprising a birnavirus as defined in the first aspect or a combination as defined in the second aspect, wherein the composition is in a form suitable for oral administration, nasal administration, or administration by inhalation.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or a nucleotide sequence according to SEQ ID NO: 2, a fragment thereof or a sequence having at least 80% sequence identity thereto.

In particular, the nucleotide sequence can be selected from the group consisting of
(i) a nucleotide sequence according to SEQ ID NO: 1 or SEQ ID NO: 2,
(ii) a nucleotide sequence that is a fragment of the nucleotide sequence according to (i) and
(iii) a nucleotide sequence that has at least 80%, preferably at least 85%, more preferably at least 90%, and most preferably at least 95% or 99%, i.e. 80, 81, 82, 83, 84, 85, 86, 87, 88, 89, 90, 91, 92, 93, 94, 95, 96, 97, 98, or 99%, sequence identity to the nucleotide sequence according to (i) or nucleotide sequence fragment according to (ii).

Preferably, the birnavirus is a live and/or attenuated birnavirus. The birnavirus may be a naturally occurring or non-naturally occurring (live and/or attenuated) birnavirus. The non-naturally occurring birnavirus is preferably a recombinant birnavirus. The non-naturally occurring birnavirus is preferably a mutated birnavirus or a chemically treated birnavirus (e.g. treated with a protease).

In one further preferred embodiment, the composition is in the form of a spray, an aerosol, tablet, dragee, capsule, solution, or suspension.

The pharmaceutical composition is for administration to a subject. The subject may be a mammal selected from the group consisting of a human being, dog, cat, sheep, goat, cow, horse, camel, and pig. It is particularly preferred that the mammal is a human being.

As to the other preferred embodiments, it is referred to the first aspect of the present invention.

In an eleventh aspect, the present invention relates to a (an in vitro) method for producing the birnavirus comprising the following steps:
(i) infecting a cell with a birnavirus,
(ii) culturing the birnavirus in the infected cell, and
(iii) isolating the cultured birnavirus or a subset of the cultured birnavirus from the infected cell.

Preferably, the cell is a cancer cell, in particular a cancer cell line. Alternatively, the cell is an immortalized cell, in particular an immortalized cell line, such as an immortalized avian cell line, e.g. the duck cell line AGE1.CR or AGE1.CR.PIX.

Thus, in an example, the method for producing the birnavirus comprises the following steps:
(i) infecting a cancer cell (line) with a birnavirus,
(ii) culturing the birnavirus in the infected cancer cell (line), and
(iii) isolating the cultured birnavirus or a subset of the cultured birnavirus from the infected cancer cell (line).

In an alternative example, the method for producing the birnavirus comprises the following steps:
(i) infecting an immortalized avian cell (line) with a birnavirus,
(ii) culturing the birnavirus in the infected immortalized avian cell (line), and
(iii) isolating the cultured birnavirus or a subset of the cultured birnavirus from the infected immortalized avian cell (line).

In a specific example, the immortalized avian cell line is the duck cell line AGE1.CR or AGE1.CR.PIX.

The cell lines AGE1.CR and AGE1.CR.PIX are commercially available from ProBioGen AG. The cell line AGE1.CR.PIX was deposited with the DSMZ-Deutsche Sammlung von Mikroorganismen und Zellkulturen GmbH, Mascheroder Weg 1b, 38124 Braunschweig, Germany on Nov. 24, 2005 under accession number DSM ACC2749.

In one preferred embodiment, the birnavirus is selected from the group consisting of an avibirnavirus, an aquabirnavirus, a blosnavirus, a dronavirus, an entomobirnavirus, a ronavirus, and a telnavirus. In one more preferred embodiment, the avibirnavirus is an Infectious Bursal Disease Virus (IBDV). In one even more preferred embodiment, the IBDV is an IBDV of strain 903/78. In one still even more preferred embodiment, the IBDV of strain 903/78 comprises a nucleotide sequence according to SEQ ID NO: 1, a fragment thereof or a sequence having at least 80% sequence identity thereto, and/or
a nucleotide sequence according to SEQ ID NO: 2, a fragment thereof or a sequence having at least 80% sequence identity thereto.

In one further preferred embodiment, the cancer cell is selected from the group consisting of a solid cancer cell, a cancer cell comprised in the hematopoietic system, and a melanoma cell. In one more preferred embodiment, the solid cancer cell is a carcinoma cell or sarcoma cell. In one alternative more preferred embodiment, the cancer cell comprised in the hematopoietic system is a lymphoma cell or leukemia cell. In one even more preferred embodiment, the carcinoma cell is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and liver carcinoma cell.

Alternatively or additionally, the cancer cell is characterized by (i) the overexpression of oral cancer overexpressed 1 (ORAOV1), voltage-dependent anion channel 2 (VDAC2), and/or receptor of activated protein kinase C1 (RACK1), (ii) cancer cells carrying a RAS mutation, and/or (iii) decreased or inhibited phosphorylation of RNA (dsRNA)-dependent protein kinase (PKR) activity.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: Induction of interferons in the human cell line A549 by infection with R903/78. Interferon beta, gamma and lambda induction as well as induction of genes downtstream of typeI IFNs ISG56 and MxA after infection with IBDV are determined comparing mRNA levels of the respective genes before and 32 h after infection at various multiplicities of infection (MOI).

FIG. 2: Enhancement of IBDV replication following treatment with 2-aminopurin in cell lines with normal RAS function (HEK293, AGE1.CR.PIX) but not in a RAS defective cell line (A549). Cells were infected with IBDV at MOI 0.05 either in the presence of 5 mM 2-aminopurin (2-AP) or without, cells together with supernatant were lysed by 3× freeze/thaw steps, cell debris was removed by centrifugation and the virus titer was quantified by TCID50.

### EXAMPLES

The examples given below are for illustrative purposes only and do not limit the invention described above in any way.

### Example 1: IBDV Rescue

Both plasmids encoding the IBDV strain R903/78 segments A (SEQ ID NO: 1) and B (SEQ ID NO: 2) were transfected into AGE1.CR.PIX cells (seeded in one well of a 6 well plate the day before) using the transfection reagent Effectene (Qiagen). Five days post transfection cells (with supernatant) were lysed by 3× freeze/thaw cycles and the lysate (1 ml) was passaged onto newly seeded AGE1.CR.PIX cells (in a T25 flask). Upon the third (identically performed) passage the TCID50 virus titer and the IBDV RNA copy numbers were quantified leading to a virus titer of 2.15×10⁶ TCID50/ml and 1.2×10⁸ IBDV copies as detected by digital droplet PCR (ddPCR). Oligonucleotides used for the ddPCR: IBDV_f: 5′-TCACTACACACTGCAGAGCA-3′ (SEQ ID NO: 3); IBDV_r: 5′-GAGACTCCGACTCACTAGCC-3′ (SEQ ID NO: 4) and the IBDV Taqman probe: IBDV_p: 5′-6FAM-TGCCCAGAACCTACCGGCCA (SEQ ID NO: 5)-BBQ-3′.

### Example 2: Manufacture of R903/78 on the AGE1.CR.PIX Cell Line

For manufacturing of IBDV R903/78, the permanent immortal cell line AGE1.CR.PIX is selected for its high permissivity and titers exceeding other virus production cell lines by 100-1000 fold reaching levels between 10⁹ and 10¹¹ infectious units.

One vial (1.5×10⁷ cells) was thawed, transferred to a shake flask grown in chemically defined culture medium CD-U5 with the addition of 10 ng/ml longR3IGF at 37° C. 5% CO₂ to a cell density of 8×10⁶/ml. For expansion, cell were diluted to 8×10⁵ cells/ml and transferred to a larger vessel grown to 8×10⁶/ml and finally seeded into the bioreactor used for infection (S.U.B, XDR disposable Stirred Tank Bioreactor, orbital shaken bioreactor) at 8×10⁵ cells/ml. For production of IBDV R903/78 cells are infected at an MOI of 0.05 when density reaches 2×10⁶/ml. Incubation is continued without feeding for 120 h. During this incubation time cells remain intact. Virus is recovered from cell culture supernatant only without destroying infected cells. Intact cells are removed by filtration with 3 μm polypropylene filters (Sartopure PP3, Sartorius, Germany) at flow rates of 170 LMH.

Titer is determined in 96 well plates seeded with 10⁵ AGE1.CR.PIX cells/well, infected with 10× serial dilutions of the virus suspension, incubated for 72 h. Infectious titer is calculated as tissue culture infection dose 50 (TCID50) using the Spearman-Karber-algorithm. The virus suspension is diluted to a final concentration of 2×10⁶, 2×10⁷, 2×10⁸, 2×10⁹ with the carbohydrate-based, stabilizing buffer (10 mM Tris base, pH 7.2 at RT, 75 mM NaCl, 1 mM MgCl₂, 0.0025% Polysorbate 80 containing 15% (w/v) sucrose). Final virus suspension was subjected to sterile filtration applying a single use 0.22 μm filter with polyvinylidenfluorid (PVDF)-membrane.

### Example 3: IBDV Induces Type I and III Interferons (IFN)

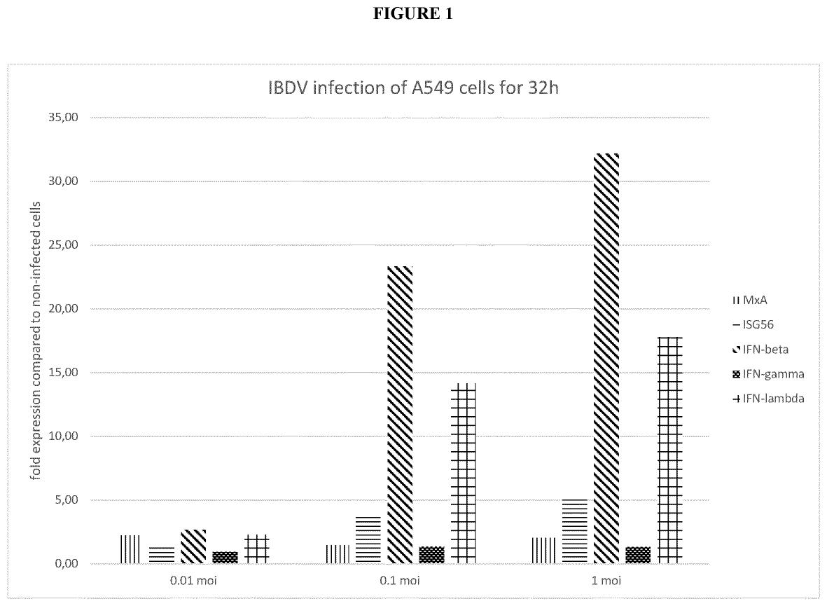

The human lung epithelial cells (A549) is typically used as an indicator cell line to analyse which type of interferons is induced by a specific virus. A549 cells are fully susceptible to infection with IBDV. To study whether type I (IFN-alpha IFN-beta), type II (IFN-gamma) and type III (IFN-lambda) are induced by IBDV, A549 cells were infected at different MOIs (0.01, 0.1 and 1) for 32 h. Subsequently, RNA was harvested out of the infected cells using the innuPREP Virus RNA kit (Analytik Jena) including a DNase digestion step. The expression levels of the different interferons (IFN-beta, IFN-gamma, IFN-lambda) and interferon-regulated genes (human myxovirus resistance protein-A (MxA) and the human interferon-stimulated gene 56 (ISG56)) were analysed by a quantitative one-step RT-PCR using the GoTaq® 1-Step RT-qPCR kit (Promega) and the following oligonucleotides: IFN-beta_f (5′-GACGCCGCATTGACCATCTA-3′ CCTTAGGATTTCCACTCTGACT-3′ SEQ ID NO: 7), IFN-lambda_f (5′-SEQ ID NO: 6). IFN-beta-r (5′-TGAGCTGGCCCTGACGCTGAA-3′ SEQ ID NO: 8), IFN-lambda_r (5′-AGGCGGAGGTTGAAGGTGA-3′ SEQ ID NO: 9), IFN-gamma_f (5′-GAAAAGCTGACTAATTATTCGGTAACTG-3′ SEQ ID NO: 10), IFN-gamma_r (5′-GTTCAGCCATCACTTGGATGAG-3′ SEQ ID NO: 11), ISG56_f (5′-CCTGGAGTACTATGAGCGGGC-3′ SEQ ID NO: 12), IFG56_r (5′-TGGGTGCCTAAGGACCTTGTC-3′ SEQ ID NO: 13), MxA_f (5′-AGGTCAGTTACCAGGACTAC-3′ SEQ ID NO: 14) and MxA_r (5′-ATGGCATTCTGGGCTTTATT-3′ SEQ ID NO: 15). The relative expression levels were normalized against non-infected cells. Glyceraldehyde-3-phosphate dehydrogenase (GAPDH) was used as an internal standard: GAPDH specific oligonucleotides GAPDH_f (5′-GGTATCGTGGAAGGACTCATGAC-3′ SEQ ID NO: 16) and GAPDH_r (5′-ATGCCAGTGAGCTTCCCGTTCAG-3′ SEQ ID NO: 17). As shown in FIG. 1, IBDV strongly induces IFN-beta in cells infected for 32 h with IBDV at multiplicity of infection 1 (MOI1) (about 30×) and IFN lambda (5-10×). In contrast to other viruses (Adenovirus, Encephalomyocarditis virus) IFN gamma is not induced.

Presence of type I IFNs is a characteristic sign of so called “hot tumors” which are infiltrated by T cells, its absence is a poor prognostic marker. Type I IFNs promote immunogenic cell death, when tumors are treated with cytotoxic cancer agents. Type I IFNs show promise in combination with chemotherapy and checkpoint inhibitor antibodies. Moreover, type I IFN induce reactivation of cancer antigens and increase HLA-class I-based antigen presentation.

IFN-lambda is a potent inducer of innate immune response, it shifts T-cell responses towards Th1 and has direct antiproliferative and pro-apoptotic action.

### Example 4: IBDV Replication is Enhanced when Cells are Treated with 2-Aminopurin that Simulates a RAS Mutation

Because susceptibility varies among cell lines, the positive impact of a RAS defect on IBDV replication has to be shown comparing cells of the same type with a specific propensity to replicate IBDV with normal and defect RAS function. Such RAS defect can be simulated in a cell line with normal RAS when cells are treated with 2-aminopurin. The drug prevents phosphorylation of protein kinase PKR that is prevented in cells with a RAS defect. The effect of 2-aminopurin on IBDV replication is studied in cell lines with (A549) and without a RAS mutation (AGE1.CR.PIX, HEK293).

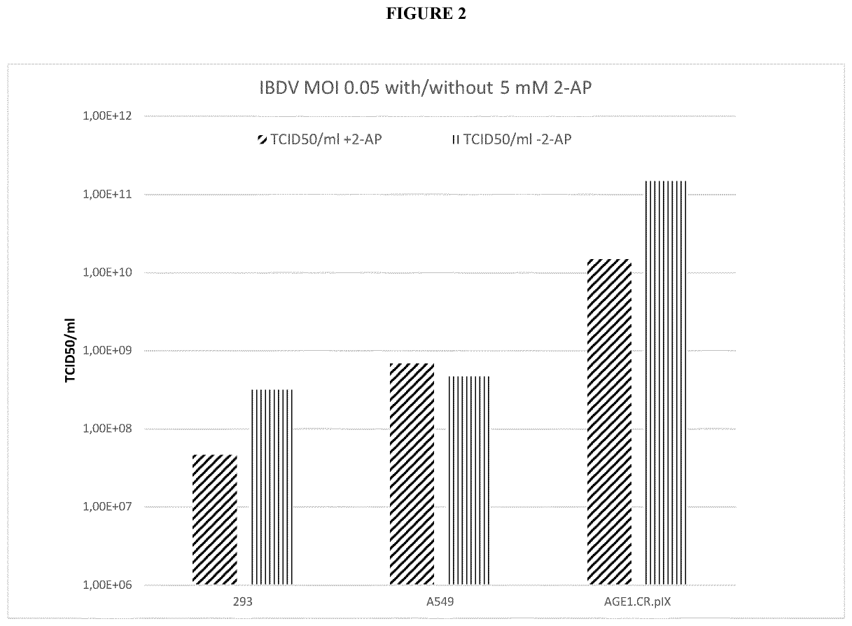

HEK 293, A549, and AGE1.CR.pIX cells were infected with IBDV at MOI 0.05 either in the presence of 5 mM 2-aminopurin (2-AP) or without. Ninety-six hours later cells (and supernatant) were lysed be 3× freeze/thaw steps and cell debris was removed by centrifugation (300×g, 5 min, room temperature). Subsequently, the virus titer was quantified by TCID50 titration in different dilutions using adherent AGE1.CR.PIX cells. Based on the cytopathic effect (CPE) the TCID50 titer was evaluated 72 hours post infection (FIG. 2).

IBDV replication is enhanced approximately 10-fold in cell lines with normal RAS (AGE1.CR.PIX, HEK293) after treatment with 2-aminopurine while no benefit (actually a titer reduction) is observed when a RAS mutant cell line (A549) is treated with the drug (A549 is fully susceptible to IBDV infection but yields are only moderate for other reasons).

### Example 5: Compassionate Use in Human Patients

### Case Report: Grade III Astrocytoma

A 28 years old male from Colorado USA was wheel-chaired into the clinic in August 2020, with right side of his body spastic, unable to walk and unable to speak, for consideration of experimental treatment options after failing all available conventional modalities. Patient developed seizures following a car accident in May 2018 and in the emergency room, a left side 7 cm mass was demonstrated. Following craniotomy, stage II anaplastic astrocytoma was diagnosed, positive for DH1 R132H, ATRX and P53 mutations, which responded well to treatment with radiation therapy and temozolomide. In January 2019 MRI confirmed tumor progression. Seizures continued and a second craniotomy in November 2019 confirmed anaplastic astrocytoma grade III. Deterioration of his condition could be controlled by additional courses of temozolomide combined with hyper-fractionated radiation, infusions of avastin and treatment with ipilimumab, pembrolizumab and nivolumab. His right arm and leg became increasingly weaker, the patient became confused, verbal skill was limited to one or two words and he became wheelchair dependent. Follow up MRI showed disease progression including crossing the midline. His physicians gave up any hope and the patient was transferred to a hospice. Being aware of the anticipated poor prognosis, the patient approached our clinic as a last resort to consider as of yet untested experimental treatment based on the use of IBDV. He was treated with oral administration of the R903/78, starting with daily increments: 10⁶, 10⁷, 10⁸ and 10⁹ infectious units with a few off-label medications and since there were no side effects, he continued treatment with daily doses of 10⁹ infectious units for 8 additional days with no side effects. It was very impressive to realize that even such high doses of R903/78, which is known to activate the native immune system (with potential anti-cancer effects) did not result in any side effects. Interestingly, by the middle of the second week of R903/78 treatment, he was walking unsupported into the clinic and his mobility was also improving, he was able to speak a few words that actually made sense, recovered his appetite and was awake and alert more than ever before. His communicative abilities-near zero on arrival-improved.

### Case Report: Grade 4 Glioblastoma

A 46 years old male from Wisconsin USA approached the clinic in September 2020 for consideration of experimental treatment of recurrent glioblastoma. Left side astrocytoma grade III was diagnosed in November 2014 following appearance of seizures. Left side awake surgery craniotomy lasting 7.5 hours was accomplished and tumor cells were characterized as astrocytic IDH1 mutated-positive MGMT positive. Patient refused additional conventional treatment with radiation and chemotherapy. Throughout 2018 the patient experienced episodes of seizures about every week and in September 2018 grand mal seizures recurred. In August 2019 disease progressed and tumor described as “fistful size” was removed in September 2019, now diagnosed as grade 4 glioblastoma. Although the surgeon was aware of residual disease, the patient again refused conventional treatment with radiation and temozolomide and instead he was searching for alternative combinations of “holistic” treatments. Due to seizures, levetiracetam and lamotrigine were added to prevent further attacks. Aphasia first and then major speech difficulties occurred associated with memory issues, he was unable to find words, had mild weakness of the right hand and he was dragging his right foot. Pre-treatment MRI report on July 2020 indicated left temporal resection cavity with moderately extensive increased T2 signal within the surrounding brain parenchyma, in the left insular cortex, left frontal lobe, and crossing the anterior commissure to the right hypothalamic region. These areas of high T2 signal are non-enhancing and do not demonstrate restricted diffusion and may represent post-therapeutic change, edema, residual non-enhancing tumor, or combination thereof. Being aware of the anticipated poor prognosis he was searching for alternative therapeutic procedures and started IBDV therapy after signing a consent form on a fully compassionate basis in September 2020. Treatment consisted of 4 incremental oral doses of R903/78: 10⁶, 10⁷, 10⁸ and 10⁹ infectious units, in parallel with several off-label medications. Treatment was well-tolerated, and patient reported no side effects and felt rather improvement of pre-treatment signs and symptoms: his speech improved and then normalized as was confirmed over a recorded Zoom interview following his return to the US. He said he was planting a Christmas tree in his garden and could even hike.

### Case Report: Cholangiocarcinoma

A 53 years old male approached the clinic for treatment of resistant stage IV hilar cholangiocarcinoma, CK7 positive, CK20 negative with confirmed peritoneal metastatic disease. During 2016 patient developed unexplained weight loss of approximately 10 kg, night pruritus. Blood tests showed elevation of liver function tests (ALP 632 IU/L; ALT 180 IU/L; AST 124 IU/L; Ferritin 499 ng/ml). Abdominal ultrasound done in October 2016 revealed normal size liver with several echogenic areas, possibly hemangiomas. In January 2017 jaundice developed. Blood test on 19 Mar. 2017 revealed bilirubin 10.3 mg/dL; ALP 1,876 IU/L; ALT 172 and AST 162 IU/L and elevated tumor marker CA19-9, 2,729 IU/mL. MRI demonstrated a mass in the bile ducts convergence area, diameter of 1.7 cm suggesting Klatskin carcinoma. Considering tumor location and potential involvement of both right and left lobes, no surgical procedure was considered, except liver transplantation. On 10 Apr. 2017 a biliary stent was introduced for drainage of rising levels of bilirubin >14 mg/dL. On 13 Apr. 2017 abdominal exploratory laparoscopy reviled peritoneal metastases and conventional chemotherapy was recommended but the patient preferred synergic combination of low doses of gemcitabine, leucovorin, fluorouracil, irinotecan and oxaliplatin administered over 48 h period every two weeks. After discovery of 2 mutations on BRCA1 (Exon 10/V9201) and BRCA2 (Exon 11/N2113S) it was decided to add PARP inhibitor and on 24 Jul. 2017 rucaparib was added and CA19-9 dropped to 287 IU/mL. CA19-9 increased following administration of nivolumab but stabilized at 250 IU/mL by adding abraxane, gemcitabine and oxaliplatin. Following addition of Erbitux and later on combinations of low-dose (1.0 mg/kg) ipilimumab and nivolumab (2 mg/kg) CA19-9 levels dropped from 369 IU/mL to normal levels. However, at the end of 2020 CA19-9 levels increased gradually up to 316-408 IU/mL towards the end of July 2020 despite the use of several new off-label medications yet accompanied by radiologic findings. Treatment with very low doses of IBDV started on 6 Aug. 2020 after signing a consent form and continued with oral IBDV R903/78 for 2 weeks starting in early September 2020 using increments from 10⁶ to 10⁹ infectious units. In parallel, immunotherapy was enhanced by down-regulation of regulatory T cells using low-dose cyclophosphamide (5 mg/kg) in combination with low-dose checkpoint inhibitors, combining ipilimumab and nivolumab. Following oral treatment with R903/78 the levels of CA19-9 dropped from a maximum of 408 to 164 IU/mL. No side effects were reported. No disease is visible by CT and MRI.

1. A method for treating or preventing cancer comprising the step of: administering a birnavirus to a subject in need thereof, thereby treating or preventing cancer in the subject.

2. The method of claim 1, wherein the birnavirus is an avibirnavirus, an aquabirnavirus, a blosnavirus, a dronavirus, an entomobirnavirus, a ronavirus, or a telnavirus.

3. The method of claim 2, wherein the avibirnavirus is an Infectious Bursal Disease Virus (IBDV).

4. The method of claim 3, wherein the IBDV is an IBDV of strain 903/78.

5-10. (canceled)

11. The method of claim 1, wherein the cancer is selected from the group consisting of a solid cancer, a cancer affecting the hematopoietic system, and a melanoma.

12. (canceled)

13. The method of claim 11, wherein the solid cancer is a carcinoma or sarcoma, and wherein
(i) the carcinoma is selected from the group consisting of a lung carcinoma, colorectal carcinoma, head and neck carcinoma, stomach carcinoma, urothelial carcinoma, breast carcinoma, cervical carcinoma, endometrial carcinoma, ovarian carcinoma, pancreatic ovarian carcinoma, brain carcinoma, prostate carcinoma, thyroid carcinoma, renal carcinoma, adrenal carcinoma, and liver carcinoma, or
(ii) the sarcoma is selected from the group consisting of a sarcoma arising in bone, muscle, fat, blood vessels, cartilage, and other soft or connective tissue of the body.

14. (canceled)

15. The method of claim 1, wherein the cancer is
(i) a cancer overexpressing oral cancer overexpressed 1 (ORAOV1), voltage-dependent anion channel 2 (VDAC2), and/or receptor of activated protein kinase C1 (RACK1),
(ii) a cancer characterized by cancer cells carrying a RAS mutation, and/or
(iii) a cancer characterized by decreased or inhibited phosphorylation of RNA (dsRNA)-dependent protein kinase (PKR) activity.

16-17. (canceled)

18. The method of claim 1, wherein the subject is receiving or having received at least one further anti-cancer therapy.

19. The method of claim 18, wherein the at least one further anti-cancer therapy is selected from the group consisting of immunomodulatory therapy, chemotherapy, immunotherapy, radiation therapy, vaccination, stem cell therapy, anti-hormonal therapy, immunosuppressive therapy, antibody therapy, and surgery.

20-21. (canceled)

22. The method of claim 1, wherein the birnavirus is administered at a dose which amounts to at least 10⁶ infectious units per day.

23. A method for treating or preventing cancer comprising the step of: administering a combination comprising at least one birnavirus and at least one further active agent suitable for the treatment or prevention of cancer to a subject in need thereof, thereby treating or preventing cancer in the subject.

24. The method of claim 23, wherein the at least one further active agent is selected from the group consisting of an immunomodulatory agent, a chemotherapeutic agent, an immunotherapeutic agent, an immunosuppressive agent, and an antibody.

25. (canceled)

26. The method of claim 23, wherein the birnavirus is administered at a dose which amounts to at least 10⁶ infectious units per day.

27-35. (canceled)

36. A kit comprising
(i) a packaging material, and
(ii) a birnavirus or a composition comprising the birnavirus, or an oral or nasal applicator, wherein the applicator comprises (a) a reservoir comprising the birnavirus or the composition, and (b) a mouth piece or nose piece, wherein the mouth piece or the nose piece is connected with/attached to the reservoir.

37. The kit of claim 36, wherein the kit further comprises
(iii) a label or packaging insert contained within the packaging material indicating that subjects
receiving the birnavirus, the combination or the pharmaceutical composition can be prevented for getting cancer or treated for cancer, or
using the reservoir or the oral or nasal applicator can be prevented for getting cancer or treated for cancer.

38. The kit of claim 37, wherein the label or packaging insert further comprises the information that the dose at which the birnavirus is to be administered amounts to at least 10⁶ infectious units per day, preferably to at least 10⁷ infectious units per day, more preferably to at least 10⁸ infectious units per day, and even more preferably to at least 10⁹ infectious units per day.

39. (canceled)

40. The method of claim 1, wherein the birnavirus is administered to the subject orally, nasally, or by inhalation.

41. A The method of claim 1, the subject is characterized as suffering from
a cancer overexpressing oral cancer overexpressed 1 (ORAOV1), voltage-dependent anion channel 2 (VDAC2), and/or receptor of activated protein kinase C1 (RACK1), a cancer comprising cancer cells carrying a RAS mutation, and/or
a cancer characterized by decreased or inhibited phosphorylation of RNA (dsRNA)-dependent protein kinase (PKR) activity.

42-43. (canceled)
